(19) United States
(12) Patent Application Publication
(10) Pub. No.: US 2009/0252783 A1
(43) Pub. Date: Oct. 8, 2009

(54) MEANS FOR INHIBITING THE EXPRESSION OF CD31

(75) Inventors: Jorg Kaufmann, Berlin (DE); Oliver Keil, Berlin (DE); Ansgar Santel, Berlin (DE)

Correspondence Address: SALIWANCHIK LLOYD & SALIWANCHIK;A PROFESSIONAL ASSOCIATION, PO Box 142950, GAINESVILLE, FL, 32614, US

(73) Assignee: SILENCE THERAPEUTICS AG, Berlin (DE)

(21) Appl. No.: 12/297,592

(22) PCT Filed: Apr. 20, 2007
(86) PCT No.: PCT/EP2007/003495

§ 371 Date: Dec. 5, 2008

(30) Foreign Application Priority Data
Apr. 20, 2006 (EP) 06008208.8

(51) Int. Cl. A61K 9/127; A61K 35/00; C12N 5/00; C12N 15/63; A61K 31/7088; C07H 21/02

(52) U.S. Cl. 424/450; 424/93.2; 435/325; 435/320.1; 514/44 R; 536/23.1

(57) ABSTRACT

The present invention is related to a nucleic acid molecule comprising a double-stranded structure, whereby the double-stranded structure comprises a first strand and a second strand, whereby the first strand comprises a first stretch of contiguous nucleotides and said first stretch is at least partially complementary to a target nucleic acid, and whereby the second strand comprises a second stretch of contiguous nucleotides and said second stretch is at least partially complementary to the first stretch, whereby the first stretch comprises a nucleic acid sequence which is at least complementary to a nucleotide core sequence of the nucleic acid sequence according to SEQ ID NO: 1, whereby the nucleotide core sequence comprises the nucleotide sequence from nucleotide positions 1277 to 1295 of SEQ ID NO: 1; from nucleotide positions 2140 to 2158 of SEQ ID NO:1; from nucleotide positions 2391 to 2409 of SEQ ID NO: 1; and whereby the first stretch is additionally at least partially complementary to a region preceding the 5′ end of the nucleotide core sequence and/or to a region following the 3′ end of the nucleotide core sequence.

The present invention is related to a double-stranded nucleic acid suitable to inhibit the expression of CD31 and use thereof.

Oncogenesis was described by Foulds (1958) as a multistep biological process, which is presently known to occur by the accumulation of genetic damage. On a molecular level, the multistep process of tumorigenesis involves the disruption of both positive and negative regulatory effectors (Weinberg, 1989). The molecular basis for human colon carcinomas has been postulated, by Vogelstein and coworkers (1990), to involve a number of oncogenes, tumor suppressor genes and repair genes. Similarly, defects leading to the development of retinoblastoma have been linked to another tumor suppressor gene (Lee et al., 1987). Still other oncogenes and tumor suppressors have been identified in a variety of other malignancies. Unfortunately, there remains an inadequate number of treatable cancers, and ‘the effects of cancer are catastrophic—over half a million deaths per year ill the United States alone.

Cancer is fundamentally a genetic disease in which damage to cellular DNA leads to disruption of the normal mechanisms that control cellular proliferation. Two of the mechanisms of action by which tumor suppressors maintain genomic integrity is by cell arrest, thereby allowing the repair of damaged DNA, or removal of the damaged DNA by apoptosis (Ellisen and Haber, 1998; Evan and Littlewood, 1998). Apoptosis, otherwise called “programmed cell death,” is a carefully regulated network of biochemical events which act as a cellular suicide program aimed at removing irreversibly damaged cells. Apoptosis can be triggered in a number of ways including binding of tumor necrosis factor, DNA damage, withdrawal of growth factors, and antibody cross-linking of Fas receptors. Although several genes have been identified that play a role in the apoptotic process, the pathways leading to apoptosis have not been fully elucidated. Many investigators have attempted to identify novel apoptosis-promoting genes with the objective that such genes would afford a means to induce apoptosis selectively in neoplastic cells to treat cancer in a patient.

An alternative approach to treating cancer involves the suppression of angiogenesis with an agent such as Endostatin™ or anti-VEGF antibodies. In this approach, the objective is to prevent further vascularization of the primary tumor and potentially to constrain the size of metastatic lesions to that which can support neoplastic cell survival without substantial vascular growth.

Platelet endothelial cell adhesion molecule which is also referred to as CD 31 or PECAM-1, is a protein found on endothelial cells and neutrophils and has been shown to be involved in the migration of leukocytes across the endothelium. The modulation of the activity of CD-31 for the treatment of cardiovascular conditions such as thrombosis, vascular occlusion and stroke and for the treatment of or for reducing blood flow obstructing diseases such as thrombosis, and for the treatment of or for reducing the occurrence of haemostasis disorders is disclosed in WO 03055516A1. PECAM-1 has also been implicated in the inflammatory process and anti-PECAM-1 monoclonal antibody has been reported to block in vivo neutrophil recruitment (Nakada et al. (2000) J. Immunol. 164: 452-462). CD31 knockout mice have been reported and appear to have normal leukocyte migration, platelet aggregation, and vascular development, which implies that there are redundant adhesion molecules which can compensate for a loss of CD31 (Duncan et al. (1999) J. Immuonol. 162: 3022-3030). Monoclonal antibodies to CD31 have been reported to block murine endothelial tube formation and related indicators of vascularization in a tumor transplantation model (Zhou et al. (1999) Angiogenesis 3: 181-188) and in a human skin transplantation model (Cao et al. (2002) Am. J. Physiol. Cell Physiol. 282: J1181-C1190). However, the role of PECAM-1 in tumor angiogenesis, if any, remains undefined.

Despite substantial efforts to inhibit cancer and the metastasis of tumors with anti-angiogenic strategies, to date there are no approved and marketed drugs for treating cancer solely by the inhibition of angiogenesis. Indeed the specific roles of various adhesion molecules, including CD31, in the processes of neoplasia and metastasis are unknown

In the lights of this, there is an ongoing need in the art for means for the treatment of neoplastic diseases. In view of the mechanisms underlying quite number of neoplastic diseases, there is more specifically a need for a means suitable to affect angiogenesis, more specifically angiogenesis involved in the pathological mechanism underlying a neoplastic disease.

The problem underlying the present invention is solved in a first aspect by a double-stranded nucleic acid molecule,
whereby the double-stranded structure comprises a first strand and a second strand,
whereby the first strand comprises a first stretch of contiguous nucleotides and said first stretch is at least partially complementary to a target nucleic acid, and
whereby the second strand comprises a second stretch of contiguous nucleotides and said second stretch is at least partially complementary to the first stretch, and
whereby the target nucleic acid is an mRNA coding for CD31.

In a preferred embodiment the nucleic acid is a ribonucleic acid.

The problem underlying the present invention is also solved in a second aspect by a nucleic acid molecule comprising a double-stranded structure,
whereby the double-stranded structure comprises a first strand and a second strand,
whereby the first strand comprises a first stretch of contiguous nucleotides and said first stretch is at least partially complementary to a target nucleic acid, and
whereby the second strand comprises a second stretch of contiguous nucleotides and said second stretch is at least partially complementary to the first stretch,
   whereby the first stretch comprises a nucleic acid sequence which is at least complementary to a nucleotide core sequence of the nucleic acid sequence according to SEQ.ID.No. 1,
whereby the nucleotide core sequence comprises the nucleotide sequence
   from nucleotide positions 1277 to 1295 of SEQ. ID.No 1;
   from nucleotide positions 2140 to 2158 of SEQ.ID.No.1;
   from nucleotide positions 2391 to 2409 of SEQ.ID.No. 1; and
      whereby the first stretch is additionally at least partially complementary to a region preceding the 5′ end of the nucleotide core sequence and/or to a region following the 3′ end of the nucleotide core sequence.

In an embodiment of the second aspect the first stretch is complementary to the nucleotide core sequence.

In an embodiment of the first and the second aspect the first stretch is additionally complementary to the region following the 3′ end of the nucleotide core sequence.

In an embodiment of the first and the second aspect the first stretch is complementary to the target nucleic acid over 18 to 29 nucleotides, preferably 19 to 25 nucleotides and more preferably 19 to 23 nucleotides.

In a preferred embodiment of the first and the second aspect the nucleotides are consecutive nucleotides.

In an embodiment of the first aspect the first stretch and/or the second stretch comprises from 18 to 29 consecutive nucleotides, preferably 19 to 25 consecutive nucleotides and more preferably 19 to 23 consecutive nucleotides.

In an embodiment of the first and the second aspect the first strand consists of the first stretch and/or the second strand consists of the second stretch.

The problem underlying the present invention is also solved in a third aspect by a nucleic acid molecule, preferably a nucleic acid molecule according to the first and the second aspect, comprising a double-stranded structure, whereby the double-stranded structure is formed by a first strand and a second one strand, whereby the first strand comprises a first stretch of contiguous nucleotides and the second strand comprises a second stretch of contiguous nucleotides and whereby said first stretch is at least partially complementary to said second stretch, whereby
the first stretch consists of a nucleotide sequence according to SEQ.ID.No. 2 and the second stretch consists of a nucleotide sequence according to SEQ.ID.No.3;
the first stretch consists of a nucleotide sequence according to SEQ.ID.No. 4 and the second stretch consists of a nucleotide sequence according to SEQ.ID.No.5;
the first stretch consists of a nucleotide sequence according to SEQ.ID.No. 6 and the second stretch consists of a nucleotide sequence according to SEQ.ID.No.7;
the first stretch consists of a nucleotide sequence according to SEQ.ID.No. 8 and the second stretch consists of a nucleotide sequence according to SEQ.ID. No. 9.

In an embodiment of the first, the second and the third aspect the first and/or the second stretch comprises a plurality of groups of modified nucleotides having a modification at the 2′ position, whereby within the stretch each group of modified nucleotides is flanked on one or both sides by a flanking group of nucleotides, whereby the flanking nucleotide(s) forming the flanking group of nucleotides is/are either an unmodified nucleotide or a nucleotide having a modification different from the modification of the modified nucleotides, whereby preferably the first stretch and/or the second stretch each comprises at least two groups of modified nucleotides and at least two flanking groups of nucleotides.

In an embodiment of the first, the second and the third aspect the first stretch and/or the second stretch comprises a pattern of groups of modified nucleotides and/or a pattern of flanking groups of nucleotides, whereby the pattern is preferably a positional pattern.

In an embodiment of the first, the second and the third aspect the first stretch and/or the second stretch comprise at the 3′ end a dinucleotide, whereby such dinucleotide is preferably TT.

In a preferred embodiment of the first, the second and the third aspect the length of the first stretch and/or of the second stretch consists of 19 to 23 nucleotides, preferably 19 to 21 nucleotides.

In an embodiment of the first, the second and the third aspect the first and/or the second stretch comprise an overhang of 1 to 5 nucleotides at the 3′ end.

In a preferred embodiment of the first, the second and the third aspect the length of the double-stranded structure is from about 16 to 24 nucleotide pairs, preferably 20 to 22 nucleotide pairs.

In an embodiment of the third aspect the first strand and the second strand are covalently linked to each other, preferably the 3′ end of the first strand is covalently linked to the 5′ end of the second strand.

The problem underlying the present invention is also solved in a fourth aspect by a lipoplex comprising a nucleic acid according to the first, the second and the third aspect and a liposome.

In an embodiment of the fourth aspect the liposome consists of
a) about 50 mol % β-arginyl-2,3-diaminopropionic acid-N-palmityl-N-oleyl-amide trihydrochloride, preferably (β-(L-arginyl)-2,3-L-diaminopropionic acid-N-palmityl-N-oleyl-amide tri-hydrochloride);
b) about 48 to 49 mol % 1,2-diphytanoyl-sn-glycero-3-phosphoethanolamine (DPhyPE); and
c) about 1 to 2 mol % 1,2-distearoyl-sn-glycero-3-phosphoethanolamine-polyethylen-glycole, preferably N-(Carbonyl-methoxypolyethyleneglycol-2000)-1,2-distearoyl-sn-glycero-3-phosphoethanolamine sodium salt.

In a preferred embodiment of the fourth aspect the zeta-potential of the lipoplex is about 40 to 55 mV, preferably about 45 to 50 mV.

In an embodiment of the fourth aspect the lipoplex has a size of about 80 to 200 nm, preferably of about 100 to 140 nm, and more preferably of about 110 nm to 130 nm, as determined by QELS.

The problem underlying the present invention is also solved in a fifth aspect by a vector, preferably an expression vector, comprising or coding for a nucleic acid according to the first, the second and the third aspect.

The problem underlying the present invention is also solved in a sixth aspect by a cell comprising a nucleic acid according to any of the preceding aspects or vector according to the fifth aspect.

The problem underlying the present invention is also solved in a seventh aspect by a composition, preferably a pharmaceutical composition, comprising a nucleic acid according to the first, the second and the third aspect, a lipoplex according to the fourth aspect, a vector according to the fifth aspect and/or a cell according to the sixth aspect.

In an embodiment of the seventh aspect the composition is a pharmaceutical composition optionally further comprising a pharmaceutically acceptable vehicle.

In a preferred embodiment of the seventh aspect the composition is a pharmaceutical composition and said pharmaceutical composition is for the treatment of an angiogenesis-dependent disease, preferably a diseases characterized or caused by insufficient, abnormal or excessive angiogenesis.

In a more preferred embodiment of the seventh aspect the angiogenesis is angiogenesis of adipose tissue, skin, heart, eye, lung, intestines, reproductive organs, bone and joints.

In an embodiment of the seventh aspect the disease is selected from the group comprising infectious diseases, autoimmune disorders, vascular malformation, atherosclerosis, transplant arteriopathy, obesity, psoriasis, warts, allergic dermatitis, persistent hyperplastic vitrous syndrome, diabetic retinopathy, retinopathy of prematurity, age-related macular disease, choroidal neovascularization, primary pulmonary hypertension, asthma, nasal polyps, inflammatory bowel and periodontal disease, ascites, peritoneal adhesions, endometriosis, uterine bleeding, ovarian cysts, ovarian, ovarian hyperstimulation, arthritis, synovitis, osteomyelitis, osteophyte formation.

In an embodiment of the seventh aspect the pharmaceutical composition is for the treatment of a neoplastic disease, preferably a cancer disease, and more preferably a solid tumor.

In an embodiment of the seventh aspect the pharmaceutical composition is for the treatment of a disease selected from the group comprising bone cancer, breast cancer, prostate cancer, cancer of the digestive system, colorectal cancer, liver cancer, lung cancer, kidney cancer, urogenital cancer, pancreatic cancer, pituitary cancer, testicular cancer, orbital cancer, head and neck cancer, cancer of the central nervous system and cancer of the respiratory system.

The problem underlying the present invention is also solved in an eighth aspect by use of a nucleic acid according to the first, the second and the third aspect, of a lipoplex according to the fourth aspect, of a vector according to the fifth aspect and/or a cell according to the sixth aspect, for the manufacture of a medicament.

In an embodiment of the eighth aspect the medicament is for the treatment of any of the diseases as defined in connection with the various embodiments of the pharmaceutical composition according to the present invention.

In a preferred embodiment of the eighth aspect the medicament is used in combination with one or several other therapies, preferably anti-tumor or anti-cancer therapies.

In a more preferred embodiment of the eighth aspect the therapy is selected from the group comprising chemotherapy, cryotherapy, hyperthermia, antibody therapy and radiation therapy.

In an even more preferred embodiment of the eighth aspect the therapy is antibody therapy and more preferably an antibody therapy using an anti-VEGF antibody.

In a further preferred embodiment of the various aspects of the present invention the mRNA is a human mRNA of CD31. In an even more preferred embodiment the target nucleic acid is an mRNA having a nucleic acid sequence in accordance with SEQ.ID.No. 1. It is to acknowledged by the ones skilled in the art that there may be one or several single nucleotide changes in the mRNA in various individuals or groups of individuals, preferably in a population, compared to the mRNA having the nucleotide sequence of SEQ.ID.No. 1. Such mRNA having one or several single nucleotide changes compared to the mRNA having a nucleic acid sequence of SEQ.ID.No. 1 shall also be comprised by the term target nucleic acid as preferably used herein. In a still further embodiment the nucleic acid molecule according to the various aspects of the invention is suitable to inhibit the expression of CD31 and the mRNA coding thereof. More preferably such expression is inhibited by a mechanism which is referred to as RNA interference or post-transcriptional gene silencing. The siRNA molecule and RNAi molecule respectively, according to the present invention is thus suitable to trigger the RNA interference response resulting preferably in the knock-down of the mRNA for the target molecule. Insofar, this kind of nucleic acid molecule is suitable to decrease the expression of a target molecule by decreasing the expression at the level of mRNA. It will be acknowledged by the one skilled in the art that there may be further mRNAs coding for CD31 which shall also be encompassed by the present application. More specifically, the particular nucleotide positions identified herein by reference to SEQ.ID.NO. 1 can be identified in such further mRNAs by the one skilled in the art based on the technical teaching provided herein.

It is also to be acknowledged that the double-stranded nucleic acid according to this aspect of the present invention may have any of the designs described herein for this kind of nucleic acid molecule. It is furthermore to be acknowledged that the mechanism described above is, in a preferred embodiment also applicable to the nucleic acids disclosed herein in connection with the various aspects and design principles also referred to herein as sub-aspects.

RNA interference refers to the process of sequence specific post-transcriptional gene silencing in animals mediated by short interfering RNAs (siRNAs) (Fire et al., 1998, Nature, 391, 806). The corresponding process in plants is commonly referred to as post-transcriptional gene silencing or RNA silencing and is also referred to as quelling in fingi. The process of post-transcriptional gene silencing is thought to be an evolutionarily-conserved cellular defence mechanism used to prevent the expression of foreign genes which is commonly shared by diverse flora and phyla (Fire et al., 1999, Trends Genet., 15, 358). Such protection from foreign gene expression may have evolved in response to the production of double-stranded RNAs (dsRNAs) derived from viral infection or the random integration of transposon elements into a host genome via a cellular response that specifically destroys homologous single-stranded RNA or viral genomic RNA. The presence of dsRNA in cells triggers the RNAi response through a mechanism that has yet to be fully characterized. This mechanism which is also existing in animal cells and in particular also in mammalian cells, appears to be different from the interferon response that results from dsRNA-mediated activation of protein kinase PKR and 2′,5′-oligoadenylate synthetase resulting in non-specific cleavage of mRNA by ribonuclease L.

The basic design of siRNA molecules or RNAi molecules, which mostly differ in the size, is basically such that the nucleic acid molecule comprises a double-stranded structure. The double-stranded structure comprises a first strand and a second strand. More preferably, the first strand comprises a first stretch of contiguous nucleotides and the second stretch comprises a second stretch of contiguous nucleotides. At least the first stretch and the second stretch are essentially complementary to each other. Such complementarity is typically based on Watson-Crick base pairing or other base-pairing mechanism known to the one skilled in the art, including but not limited to Hoogsteen base-pairing and others. It will be acknowledged by the one skilled in the art that depending on the length of such double-stranded structure a perfect match in terms of base complementarity is not necessarily required. However, such perfect complementarity is preferred in some embodiments. In a particularly preferred embodiment the complementarity and/or identity is at least 75%, 80%, 85%, 90% or 95%. In an alternative particularly preferred embodiment, the complementarity and/or identity is such that the complement and/or identical nucleic acid molecule hybridizes to one of the strands of the nucleic acid molecule according to the present invention, more preferably to one of the two stretches under the following conditions: is capable of hybridizing with a portion of the target gene transcript under the following conditions: 400 mM NaCl, 40 mM PIPES pH 6.4, 1 mM EDTA, 50° C. or 70° C. hybridisation for 12-16 hours, followed by washing. Respective reactions conditions are, among others described in European patent EP 1 230 375. In any case, the nucleic acid molecules according to the present invention are designed or embodied such that they are suitable for gene silencing and more specifically suitable to trigger RNA interference.

A mismatch is also tolerable, mostly under the proviso that the double-stranded structure is still suitable to trigger the RNA interference mechanism, and that preferably such double-stranded structure is still stably forming under physiological conditions as prevailing in a cell, tissue and organism, respectively, containing or in principle containing such cell, tissue and organ. More preferably, the double-stranded structure is stable at 37° C. in a physiological buffer. It will be acknowledged by the ones skilled in the art that this kind of mismatch can preferably be contained at a position within the nucleic acid molecule according to the present invention different from the core region.

The first stretch, is typically at least partially complementary to a target nucleic acid and the second stretch is, particularly given the relationship between the first and second stretch, respectively, in terms of base complementarity, at least partially identical to the target nucleic acid. The target nucleic acid is preferably an mRNA, although other forms of RNA such as hnRNAs are also suitable for the purpose of the nucleic acid molecule as disclosed herein.

Although RNA interference can be observed upon using long nucleic acid molecules comprising several dozens and sometimes even several hundreds of nucleotides and nucleotide pairs, respectively, shorter RNAi molecules are generally preferred. A more preferred range for the length of the first stretch and/or second stretch is from about 18 to 29 consecutive nucleotides, preferably 19 to 25 consecutive nucleotides and more preferably 19 to 23 consecutive nucleotides. More preferably, both the first stretch and the second stretch have the same length. In a further embodiment, the double-stranded structure comprises preferably between 16 and 29, preferably 18 to 25, more preferably 19 to 23 and most preferably 19 to 21 base pairs.

Although in accordance with the present invention, in principle, any part of the mRNA coding for CD31 can be used for the design of such siRNA molecule and RNAi molecule, respectively, the present inventors have surprisingly found that the sequence starting with nucleotide positions 1277, 2140, and 2391 of the mRNA of SEQ.ID.NO. 1 having the nucleotide sequence of SEQ.ID.No.1 are particularly suitable to be addressed by RNA interference mediating molecule:

More specifically, the present inventors have surprisingly found that although these sequences and starting points are particularly preferred target sequence for expression inhibition of CD31, there is a core of nucleotides in the vicinity of these sequences which is particularly effective insofar. This core is in one embodiment a sequence consisting of the about 9 to 11 last nucleotides of the above specified nucleotide sequences. Starting therefrom, the core can be extended such that a functionally active double-stranded nucleic acid molecule is obtained, whereby preferably functionally active means suitable to affect expression inhibition of CD31. For such purpose, the second stretch which is essentially identical to the corresponding part of the mRNA, i.e. the core sequence, is thus prolonged by one, preferably several nucleotides at the 5′ end, whereby the thus added nucleotides are essentially identical to the nucleotides present in the target nucleic acid at the corresponding positions. Also for such purpose, the first strand which is essentially complementary to the target nucleic acid, is thus prolonged by one, preferably several nucleotides at the 3′ end, whereby the thus added nucleotides are essentially complementary to the nucleotides present in the target nucleic acid at the corresponding positions, i.e. at the 5′ end.

In accordance with this design principle, the core sequences according to the present invention can be summarized as follows:

5′cauccagaa3′,
5′acuccaaca3′,
and
5′agaacggaa3′

In a further embodiment thereof, the core sequence is identical to the nucleotide sequence of the second stretch of the double-stranded nucleic acid molecule according to the present invention and the first stretch essentially complementary thereto. In a still further preferred embodiment, the length of the double-stranded nucleic acid molecule according to the present invention is within the limits disclosed herein in connection with the various aspects and sub-aspects, respectively.

It will be acknowledged by the ones skilled in the art that the particular design of the siRNA molecules and the RNAi molecules, respectively, can vary in accordance with the current and future design principles. For the time being some design principles exist which shall be discussed in more detail in the following and which shall be referred to as sub-aspects or sub-aspects of the first aspect of the nucleic acid molecule according to the present invention. It is within the present invention that all features and embodiments described for one particular sub-aspect, i.e. design of the nucleic acid, are also applicable to any other aspect and sub-aspect of the nucleic acid according to the present invention and thus form respective embodiments thereof.

The first sub-aspect is related to nucleic acid according to the present invention, whereby the first stretch comprises a plurality of groups of modified nucleotides having a modification at the 2′ position, whereby within the stretch each group of modified nucleotides is flanked on one or both sides by a flanking group of nucleotides, whereby the flanking nucleotide(s) forming the flanking group(s) of nucleotides is either an unmodified nucleotide or a nucleotide having a modification different from the modification of the modified nucleotides. Such design is, among others described in international patent application WO 2004/015107. The nucleic acid according to this aspect is preferably a ribonucleic acid although, as will be outlined in some embodiments, the modification at the 2′ position results in a nucleotide which as such is, from a pure chemical point of view, no longer a ribonucleotide. However, it is within the present invention that such modified ribonucleotide shall be regarded and addressed herein as a ribonucleotide and the molecule containing such modified ribonucleotide as a ribonucleic acid.

In an embodiment of the ribonucleic acid according to the first sub-aspect of the present invention the ribonucleic acid is blunt ended, either on one side or on both sides of the double-stranded structure. In a more preferred embodiment the double-stranded structure comprises 18 to 25, more preferably 19 to 23 and, alternatively, 18 or 19 base pairs. In an even more preferred embodiment, the nucleic acid consists of the first stretch and the second stretch only.

In a further embodiment of the ribonucleic acid according to the first sub-aspect of the present invention said first stretch and/or said second stretch comprise a plurality of groups of modified nucleotides. In a further preferred embodiment the first stretch also comprises a plurality of flanking groups of nucleotides. In a preferred embodiment a plurality of groups means at least two groups.

In another embodiment of the ribonucleic acid according to the first sub-aspect of the present invention said second stretch comprises a plurality of groups of modified nucleotides. In a further preferred embodiment the second stretch also comprises a plurality of flanking groups of nucleotides. In a preferred embodiment a plurality of groups means at least two groups.

In a further preferred embodiment both the first and the second stretch comprise a plurality of both groups of modified nucleotides and flanking groups of nucleotides. In a more preferred embodiment the plurality of both groups of modified nucleotides and flanking groups of nucleotides form a pattern, preferably a regular pattern, on either the first stretch and/or the second stretch, whereby it is even more preferred that such pattern is formed on both the first and the second stretch. In a preferred embodiment such pattern is a spatial or positional pattern. A spatial or positional pattern as subject to this first sub-aspect means that (a) nucleotide(s) is/are modified dependent on the position within the nucleotide sequence of a strand/stretch forming the double-stranded structure. Accordingly, it does not matter whether the nucleotide to be modified is a pyrimidine or a purine. Rather the relative position of such nucleotide(s) relative to (a) non-modified nucleotide(s) and thus relative to the 5′ end and the 3′ end, respectively, is decisive insofar. Therefore, the modification(s) seen along the individual strand/stretch is thus not dependent on or even driven by the chemical nature of the individual nucleotide along such strand/stretch, but depends on the position of the individual nucleotide. Therefore, according to the technical teaching of this first sub-aspect of the present invention, the modification pattern will always be the same, irrespective of the sequence which is to be modified.

In a preferred embodiment of the ribonucleic acid according to the first sub-aspect of the present invention the group of modified nucleotides and/or the group of flanking nucleotides comprises a number of nucleotides whereby the number is selected from the group comprising one nucleotide to 10 nucleotides. In connection with any ranges specified herein it is to be understood that each range discloses any individual integer between the respective figures used to define the range including said two figures defining said range. In the present case the group thus comprises one nucleotide, two nucleotides, three nucleotides, four nucleotides, five nucleotides, six nucleotides, seven nucleotides, eight nucleotides, nine nucleotides and ten nucleotides.

In another embodiment of the ribonucleic acid according to the first sub-aspect of the present invention the pattern of modified nucleotides of said first stretch is the same as the pattern of modified nucleotides of said second stretch.

In a preferred embodiment of the ribonucleic acid according to the first sub-aspect of the present invention the pattern of said first stretch aligns with the pattern of said second stretch.

In an alternative embodiment of the ribonucleic acid according to the first sub-aspect of the present invention the pattern of said first stretch is shifted by one or more nucleotides relative to the pattern of the second stretch.

In an embodiment of the ribonucleic acid according to the first sub-aspect of the present invention the modification at the 2′ position is selected from the group comprising amino, fluoro, methoxy, alkoxy and alkyl.

In another embodiment of the ribonucleic acid according to the first sub-aspect of the present invention the double stranded structure is blunt ended.

In a preferred embodiment of the ribonucleic acid according to the first sub-aspect of the present invention the double stranded structure is blunt ended on both sides of the double-stranded structure.

In another embodiment of the ribonucleic acid according to the first sub-aspect of the present invention the double stranded structure is blunt ended on the double stranded structure's side which is defined by the 5′-end of the first strand and the 3′-end of the second strand.

In still another embodiment of the ribonucleic acid according to the first sub-aspect of the present invention the double stranded structure is blunt ended on the double stranded structure's side which is defined by at the 3′-end of the first strand and the 5′-end of the second strand.

In another embodiment of the ribonucleic acid according to the first sub-aspect of the present invention at least one of the two strands has an overhang of at least one nucleotide at the 5′-end.

In a preferred embodiment of the ribonucleic acid according to the first sub-aspect of the present invention the overhang consists of at least one deoxyribonucleotide.

In a further embodiment of the ribonucleic acid according to the first sub-aspect of the present invention at least one of the strands has an overhang of at least one nucleotide at the 3′-end.

In an embodiment of the ribonucleic acid of the first sub-aspect the length of the double-stranded structure is from about 17 to 25, and more preferably 19 to 23 base pairs or 18 or 19 base pairs.

In another embodiment of the ribonucleic acid of the first sub-aspect the length of said first strand and/or the length of said second strand is independently from each other selected from the group comprising the ranges of from about 15 to about 23 base pairs, 19 to 23 base pairs and 18 or 19 base pairs.

In a preferred embodiment of the ribonucleic acid according to the first sub-aspect the present invention the complementarity between said first strand and the target nucleic acid is perfect.

In an embodiment of the ribonucleic acid according to the first sub-aspect the duplex formed between the first strand and the target nucleic acid comprises at least 15 nucleotides wherein there is one mismatch or two mismatches between said first strand and the target nucleic acid forming said double-stranded structure.

In an embodiment of the ribonucleic acid according to the first sub-aspect both the first strand and the second strand each comprise at least one group of modified nucleotides and at least one flanking group of nucleotides, whereby each group of modified nucleotides comprises at least one nucleotide and whereby each flanking group of nucleotides comprising at least one nucleotide with each group of modified nucleotides of the first strand being aligned with a flanking group of nucleotides on the second strand, whereby the most terminal 5′ nucleotide of the first strand is a nucleotide of the group of modified nucleotides, and the most terminal 3′ nucleotide of the second strand is a nucleotide of the flanking group of nucleotides. In a preferred embodiment, the first strand and the second strand each comprise at lest two groups of modified nucleotides and at least two groups of flanking groups of nucleotides. In a still more preferred embodiment each and any individual group consists of a single nucleotide.

In a preferred embodiment of the ribonucleic acid according to of the first sub-aspect, each group of modified nucleotides consists of a single nucleotide and/or each flanking group of nucleotides consists of a single nucleotide.

In a further embodiment of the ribonucleic acid according to of the first sub-aspect, on the first strand the nucleotide forming the flanking group of nucleotides is an unmodified nucleotide which is arranged in a 3′ direction relative to the nucleotide forming the group of modified nucleotides, and wherein on the second strand the nucleotide forming the group of modified nucleotides is a modified nucleotide which is arranged in 5′ direction relative to the nucleotide forming the flanking group of nucleotides.

In another embodiment of the ribonucleic acid according to the first sub-aspect, the first strand comprises eight to twelve, preferably nine to eleven, groups of modified nucleotides, and wherein the second strand comprises seven to eleven, preferably eight to ten, groups of modified nucleotides.

It is within the present invention that what has been specified above is also applicable to the first and second stretch, respectively. This is particular true for those embodiments where the strand consists of the stretch only.

The ribonucleic acid molecule according to such first sub-aspect may be designed is to have a free 5′ hydroxyl group, also referred to herein as free 5′ OH-group, at the first strand. A free 5′ OH-group means that the most terminal nucleotide forming the first strand is present and is thus not modified, particularly not by an end modification. Typically, the terminal 5′-hydroxy group of the second strand, respectively, is also present in an unmodified manner. In a more preferred embodiment, also the 3′-end of the first strand and first stretch, respectively, is unmodified such as to present a free OH-group which is also referred to herein as free 3′ OH-group, whereby the design of the 5′ terminal nucleotide is the one of any of the aforedescribed embodiments. Preferably such free OH-group is also present at the 3′-end of the second strand and second stretch, respectively. In other embodiments of the ribonucleic acid molecules as described previously according to the present invention the 3′-end of the first strand and first stretch, respectively, and/or the 3′-end of the second strand and second stretch, respectively, may have an end modification at the 3′ end.

As used herein the terms free 5′ OH-group and 3′ OH-group also indicate that the respective most terminal nucleotide at the 5′ end and the 3′ end of the polynucleotide, respectively, i.e. either the nucleic acid or the strands and stretches, respectively, forming the double-stranded structure present an OH-group. Such OH-group may stem from either the sugar moiety of the nucleotide, more preferably from the 5′position in case of the 5′ OH-group and/or from the 3′ position in case of the 3′ OH-group, or from a phosphate group attached to the sugar moiety of the respective terminal nucleotide. The phosphate group may in principle be attached to any OH-group of the sugar moiety of the nucleotide. Preferably, the phosphate group is attached to the 5′ OH-group of the sugar moiety in case of the free 5′ OH-group and/or to the 3′ OH-group of the sugar moiety in case of the free 3′ OH-group still providing what is referred to herein as free 5′ OH-group or 3′ OH-group.

As used herein with any embodiment of the first sub-aspect, the term end modification means a chemical entity added to the most 5′ or 3′ nucleotide of the first and/or second strand. Examples for such end modifications include, but are not limited to, inverted (deoxy) abasics, amino, fluoro, chloro, bromo, CN, CF, methoxy, imidazole, carboxylate, thioate, C₁ to C₁₀ lower alkyl, substituted lower alkyl, alkaryl or aralkyl, OCF₃, OCN, O-, S-, or N-alkyl; O-, S-, or N-alkenyl; SOCH₃; SO₂CH₃; ONO₂; NO₂, N₃; heterozycloalkyl; heterozycloalkaryl; aminoalkylamino; polyalkylamino or substituted silyl, as, among others, described in European patents EP 0 586 520 B1 or EP 0 618 925 B1.

As used herein, alkyl or any term comprising “alkyl” means any carbon atom chain comprising 1 to 12, preferably 1 to 6 and more, preferably 1 to 2 C atoms.

A further end modification is a biotin group. Such biotin group may preferably be attached to either the most 5′ or the most 3′ nucleotide of the first and/or second strand or to both ends. In a more preferred embodiment the biotin group is coupled to a polypeptide or a protein. It is also within the scope of the present invention that the polypeptide or protein is attached through any of the other aforementioned end modifications. The polypeptide or protein may confer further characteristics to the inventive nucleic acid molecules. Among others the polypeptide or protein may act as a ligand to another molecule. If said other molecule is a receptor the receptor's function and activity may be activated by the binding ligand. The receptor may show an internalization activity which allows an effective transfection of the ligand bound inventive nucleic acid molecules. An example for the ligand to be coupled to the inventive nucleic acid molecule is VEGF and the corresponding receptor is the VEGF receptor.

Various possible embodiments of the RNAi of the present invention having different kinds of end modification(s) are presented in the following table 1.

TABLE 1
Various embodiments of the interfering ribonucleic
acid according to the present invention

| | | 1^{st} strand/1^{st} stretch | 2^{nd} strand/2nd stretch |
|---|---|---|---|
| 1.) | 5′-end | free OH | free OH |
| | 3′-end | free OH | free OH |
| 2.) | 5′-end | free OH | free OH |
| | 3′-end | end modification | end modification |
| 3.) | 5′-end | free OH | free OH |
| | 3′-end | free OH | end modification |
| 4.) | 5′-end | free OH | free OH |
| | 3′-end | end modification | free OH |
| 5.) | 5′-end | free OH | end modification |
| | 3′-end | free OH | free OH |
| 6.) | 5′-end | free OH | end modification |
| | 3′-end | end modification | free OH |
| 7.) | 5′-end | free OH | end modification |
| | 3′-end | free OH | end modification |
| 8.) | 5′-end | free OH | end modification |
| | 3′-end | end modification | end modification |

The various end modifications as disclosed herein are preferably located at the ribose moiety of a nucleotide of the ribonucleic acid. More particularly, the end modification may be attached to or replace any of the OH-groups of the ribose moiety, including but not limited to the 2′ OH, 3′ OH and 5′ OH position, provided that the nucleotide thus modified is a terminal nucleotide. Inverted abasics are nucleotides, either desoxyribonucleotides or ribonucleotides which do not have a nucleobase moiety. This kind of compound is, among others, described in Sternberger et al., 2002.

Any of the aforementioned end modifications may be used in connection with the various embodiments of RNAi depicted in table 1. In connection therewith it is to be noted that any of the RNAi forms or embodiments disclosed herein with the sense strand being inactivated, preferably by having an end modification, more preferably at the 5′ end, are particularly advantageous. This arises from the inactivation of the sense strand which corresponds to the second strand of the ribonucleic acids described herein, which might otherwise interfere with an unrelated single-stranded RNA in the cell. Thus the expression and more particularly the translation pattern of the transcriptome of a cell is more specifically influenced. This effect is also referred to as off-target effect. Referring to table 1 those embodiments depicted as embodiments 7 and 8 are particularly advantageous in the above sense as the modification results in an inactivation of the—target unspecific—part of the RNAi (which is the second strand) thus reducing any unspecific interaction of the second strand with single-stranded RNA in a cellular or similar system where the RNAi according to the present invention is going to be used to knock down specific ribonucleic acids and proteins, respectively.

In a further embodiment, the nucleic acid according to the first sub-aspect has an overhang at the 5′-end of the ribonucleic acid. More particularly, such overhang may in principle be present at either or both the first strand and second strand of the ribonucleic acid according to the present invention. The length of said overhang may be as little as one nucleotide and as long as 2 to 8 nucleotides, preferably 2, 4, 6 or 8 nucleotides. It is within the present invention that the 5′ overhang may be located on the first strand and/or the second strand of the ribonucleic acid according to the present application. The nucleotide(s) forming the overhang may be (a) desoxyribonucleotide(s), (a) ribonucleotide(s) or a combination thereof.

The overhang preferably comprises at least one desoxyribonucleotide, whereby said one desoxyribonucleotide is preferably the most 5′-terminal one. It is within the present invention that the 3′-end of the respective counter-strand of the inventive ribonucleic acid does not have an overhang, more preferably not a desoxyribonucleotide overhang. Here again, any of the inventive ribonucleic acids may comprise an end modification scheme as outlined in connection with table 1 and/or an end modification as outlined herein.

Taken the stretch of contiguous nucleotides a pattern of modification of the nucleotides forming the stretch may be realised in an embodiment such that a single nucleotide or group of nucleotides which are covalently linked to each other via standard phosphorodiester bonds or, at least partially, through phosphorothioate bonds, show such kind of modification. In case such nucleotide or group of nucleotides which is also referred to herein as group of modified nucleotides, is not forming the 5′-end or 3′-end of said stretch a nucleotide or group of nucleotides follows on both sides of the nucleotide which does not have the modification of the preceding nucleotide or group of nucleotides. It is to be noted that this kind of nucleotide or group of nucleotides, however, may have a different modification. This kind of nucleotide or group of nucleotides is also referred to herein as the flanking group of nucleotides. This sequence consisting of modified nucleotide and group(s) of modified nucleotides, respectively, and of unmodified or differently modified nucleotide or group(s) of unmodified or differently modified nucleotides may be repeated one or several times. Preferably, the sequence is repeated more than one time. For reason of clarity the pattern is discussed in more detail in the following, generally referring to a group of modified nucleotides or a group of unmodified nucleotides whereby each of said groups may actually comprise as little as a single nucleotide. Unmodified nucleotide as used herein means either not having any of the afore-mentioned modifications at the nucleotide forming the respective nucleotide or group of nucleotides, or having a modification which is different from the one of the modified nucleotide and group of nucleotides, respectively.

It is also within the present invention that the modification of the unmodified nucleotide(s) wherein such unmodified nucleotide(s) is/are actually modified in a way different from the modification of the modified nucleotide(s), can be the same or even different for the various nucleotides forming said unmodified nucleotides or for the various flanking groups of nucleotides.

The pattern of modified and unmodified nucleotides may be such that the 5′-terminal nucleotide of the strand or of the stretch starts with a modified group of nucleotides or starts with an unmodified group of nucleotides. However, in an alternative embodiment it is also possible that the 5′-terminal nucleotide is formed by an unmodified group of nucleotides.

This kind of pattern may be realised either on the first stretch or the second stretch of the interfering RNA or on both. This applies equally to the first strand and the second strand, respectively. It has to be noted that a 5′ phosphate on the target-complementary strand of the siRNA duplex is required for siRNA function, suggesting that cells check the authenticity of siRNAs through a free 5′ OH (which can be phosphorylated) and allow only such bona fide siRNAs to direct target RNA destruction (Nykanen, 2001 #94).

Preferably, the first stretch shows a kind of pattern of modified and unmodified groups of nucleotides, i.e. of group(s) of modified nucleotides and flanking group(s) of nucleotides, whereas the second stretch does not show this kind of pattern. This may be useful insofar as the first stretch is actually the more important one for the target-specific degradation process underlying the interference phenomenon of RNA so that for specificity reasons the second stretch can be chemically modified so it is not functional in mediating RNA interference. This applies equally to the first strand and the second strand, respectively.

However, it is also within the present invention that both the first stretch and the second stretch have this kind of pattern. Preferably, the pattern of modification and non-modification is the same for both the first stretch and the second stretch. This applies equally to the first strand and the second strand, respectively.

In a preferred embodiment the group of nucleotides forming the second stretch and corresponding to the modified group of nucleotides of the first stretch are also modified whereas the unmodified group of nucleotides of or forming the second stretch correspond to the unmodified group of nucleotides of or forming the first stretch. Another alternative is that there is a phase shift of the pattern of modification of the first stretch and first strand, respectively, relative to the pattern of modification of the second stretch and second strand, respectively. Preferably, the shift is such that the modified group of nucleotides of the first stretch corresponds to the unmodified group of nucleotides of the second stretch and vice versa. It is also within the present invention that the phase shift of the pattern of modification is not complete but overlapping. This applies equally to the first strand and the second strand, respectively.

In a preferred embodiment the second nucleotide at the terminus of the strand and stretch, respectively, is an unmodified nucleotide or the beginning of group of unmodified nucleotides. Preferably, this unmodified nucleotide or unmodified group of nucleotides is located at the 5′-end of the first and second strand, respectively, and even more preferably of the first strand. In a further preferred embodiment the unmodified nucleotide or unmodified group of nucleotide is located at the 5′-end of the first strand and first stretch, respectively. In a preferred embodiment the pattern consists of alternating single modified and unmodified nucleotides.

In a further preferred embodiment of this aspect of the present invention the interfering ribonucleic acid subject comprises two strands, whereby a 2′-O-methyl modified nucleotide and a non-modified nucleotide, preferably a nucleotide which is not 2′-O-methyl modified, are incorporated on both strands in an alternate manner which means that every second nucleotide is a 2′-O-methyl modified and a non-modified nucleotide, respectively. This means that on the first strand one 2′-O-methyl modified nucleotide is followed by a non-modified nucleotide which in turn is followed by 2′-O-methyl modified nucleotide and so on. The same sequence of 2′-O-methyl modification and non-modification exists on the second strand, whereby there is preferably a phase shift such that the 2′-O-methyl modified nucleotide on the first strand base pairs with a non-modified nucleotide(s) on the second strand and vice versa. This particular arrangement, i.e. base pairing of 2′-O-methyl modified and non-modified nucleotide(s) on both strands is particularly preferred in case of short interfering ribonucleic acids, i.e. short base paired double-stranded ribonucleic acids because it is assumed, although the present inventors do not wish to be bound by that theory, that a certain repulsion exists between two base-pairing 2′-O-methyl modified nucleotides which would destabilise such duplex, and short duplexes in particular. About the particular arrangement, it is preferred if the antisense strand starts with a 2′-O-methyl modified nucleotide at the 5′ end whereby consequently the second nucleotide is non-modified, the third, fifth, seventh and so on nucleotides are thus again 2′-O-methyl modified whereas the second, fourth, sixth, eighth and the like nucleotides are non-modified nucleotides. Again, not wishing to be bound by any theory, it seems that particular importance may be ascribed to the second, and optionally fourth, sixth, eighth and/or similar position(s) at the 5′ terminal end of the antisense strand which should not comprise any modification, whereas the most 5′ terminal nucleotide, i.e. the first 5′ terminal nucleotide of the antisense strand may exhibit such modification with any uneven positions such as first, optionally third, fifth and similar position(s) at or of the antisense strand may be modified. In further embodiments the modification and non-modification, respectively, of the modified and non-modified nucleotide(s), respectively, may be anyone as described herein. In a more specific embodiment, the double-stranded nucleic acid molecule according to the present invention consists of a first strand of 19 to 23 consecutive nucleotides and a second strand of 19 to 23 consecutive nucleotides, whereby the first strand and the second strand are essentially complementary to each other and more preferably have the same length. Furthermore, in said more specific embodiment the double-stranded structure is blunt-ended at both end. The first strand which is essentially complementary to the target nucleic acid, i.e. an mRNA coding for CD31, starts at the 5′ end with a nucleotide which is methylated at the 2′OH group forming a 2′O-Me group. Every second nucleotide of this first strand has the same modification, i.e. is methylated at the 2′ OH group. Thus, the first, third, fifth and so on, i.e. any uneven nucleotide position of the first strand is modified in such a way. The nucleotides at the even positions of the first strand are either non-modified nucleotides or modified nucleotides, whereby if modified, the modification is different from the modification of the nucleotides at the uneven nucleotide positions of the first strand. The second strand preferably comprising the same number of nucleotides as the first strand, has a modified nucleotide at the second, fourth, sixth and so on, i.e. at any even nucleotide position when counting in contrast to the usual counting direction herein, which is 5′->3′, from or in 3′->5′ direction. Any of the other nucleotides, i.e. those at the uneven nucleotide positions are non-modified nucleotides or modified nucleotides, whereby if modified, the modification is different from the modification of the nucleotides at the even nucleotide positions of the second strand. Therefore the second strand starts at the 5′ end with a non-modified nucleotide in the above sense. In a more preferred embodiment, the modification of the modified nucleotides of the first and the second strand is the same and the modification of the non-modified nucleotides of the first and the second strand is also the same. In a preferred embodiment the 5′ end of the antisense strand has a OH-group which preferably may be phosphorylated in a cell, preferably in a target cell, where the nucleic acid molecule of the present invention is to be active or functional, or has a phosphate group. The 5′ end of the sense strand is preferably also modified, more preferably modified as disclosed herein. Any or both of the 3′ ends have, in an embodiment, a terminal phosphate.

It is within the present invention that the double-stranded structure is formed by two separate strands, i.e. the first and the second strand. However, it is also with in the present invention that the first strand and the second strand are covalently linked to each other. Such linkage may occur between any of the nucleotides forming the first strand and second strand, respectively. However, it is preferred that the linkage between both strands is made closer to one or both ends of the double-stranded structure. Such linkage can be formed by covalent or non-covalent linkages. Covalent linkage may be formed by linking both strands one or several times and at one or several positions, respectively, by a compound preferably selected from the group comprising methylene blue and bifunctinoal groups. Such bifunctional groups are preferably selected from the group comprising bis(2-chloroethyl)amine, N-acetyl)-N′-(p-glyoxylbenzoyl)cystamine, 4-thiouracile and psoralene.

In a further embodiment of the ribonucleic acid according to any of the first sub-aspects of the present invention the first strand and the second strand are linked by a loop structure.

In a preferred embodiment of the ribonucleic acid according to the first sub-aspects of the present invention the loop structure is comprised of a non-nucleic acid polymer.

In a preferred embodiment thereof the non-nucleic acid polymer is polyethylene glycol.

In an embodiment of the ribonucleic acid according to any of the first sub-aspects of the present invention the 5′-terminus of the first strand is linked to the 3′-terminus of the second strand.

In a further embodiment of the ribonucleic acid according to any of the aspects of the present invention the 3′-end of the first strand is linked to the 5′-terminus of the second strand.

In an embodiment the loop consists of a nucleic acid. As used herein, LNA as described in Elayadi and Corey (2001) Curr Opin Investig Drugs. 2(4):558-61. Review; Orum and Wengel (2001) Curr Opin Mol Ther. 3(3):239-43; and PNA are regarded as nucleic acids and may also be used as loop forming polymers. Basically, the 5′-terminus of the first strand may be linked to the 3′-terminus of the second strand. As an alternative, the 3′-end of the first strand may be linked to the 5′-terminus of the second strand. The nucleotide sequence forming said loop structure is regarded as in general not being critical. However, the length of the nucleotide sequence or the units forming such nucleotide sequence which in turn forms such loop seems to be critical for sterical reasons. Accordingly, a minimum length of four nucleotides or nucleotide analogues seems to be appropriate to form the required loop structure. In principle, the maximum number of nucleotides forming the hinge or the link between both stretches or strands to be hybridised is not limited. However, the longer a polynucleotide is, the more likely secondary and tertiary structures are formed and thus the required orientation of the stretches affected. Preferably, a maximum number of nucleotides forming the hinge is about 12 nucleotides or nucleotide analogues. It is within the disclosure of this application that any of the designs described above may be combined with any of the other designs disclosed herein and known in the art, respectively, i.e. by linking the two strands covalently in a manner that a back folding can occur through a loop structure or similar structure.

The present inventors have surprisingly found that if the loop is placed 3′ of the antisense strand, i.e. the first strand of the ribonucleic acid(s) according to the present invention, the activities of this kind of RNAi are higher compared to the placement of the loop 5′ of the antisense strand. Accordingly, the particular arrangement of the loop relative to the antisense strand and sense strand, i.e. the first strand and the second strand, respectively, is crucial and is thus in contrast to the understanding as expressed in the prior art where the orientation is said to be of no relevance. However, this seems not true given the experimental results presented herein. The understanding as expressed in the prior art is based on the assumption that any RNAi is subject to a processing during which non-loop linked RNAi is generated. However, if this was the case, the clearly observed increased activity of those structures having the loop placed 3′ of the antisense could not be explained. Insofar a preferred arrangement in 5′→3′ direction of this kind of small interfering RNAi is second strand-loop-first strand. The respective constructs may be incorporated into suitable vector systems. Preferably the vector comprises a promoter for the expression of RNAi. Preferably the respective promoter is pol III and more preferably the promoters are the U6, Hi, 7SK promoter as described in Good et al. (1997) Gene Ther, 4, 45-54.

The second sub-aspect of the first aspect of the present invention is related to a nucleic acid according to the present invention, whereby the first stretch and/or the second stretch comprise at the 3′ end a dinucleotide, whereby such dinucleotide is preferably TT. In a preferred embodiment, the length of the first stretch and/or of the second stretch consists of 18 to 23 nucleotides and more preferably the double-stranded structure comprises 18 to 23 and more preferably 19 to 21 base pairs. The design of the nucleic acid in accordance with this sub-aspect is described in more detail in, e.g., in international patent application WO 01/75164.

The third sub-aspect of the first aspect of the present invention is related to a nucleic acid according to the present invention, whereby the first and/or the second stretch comprise an overhang of 1 to 5 nucleotides at the 3′ end. The design of the nucleic acid in accordance with this sub-aspect is described in more detail in international patent application WO02/44321. More preferably such overhang is a ribonucleic acid. In a preferred embodiment each of the strands and more preferably each of the stretches as defined herein has a length from 19 to 25 nucleotides, whereby more preferably the strand consists of the stretch. In a preferred embodiment, the double-stranded structure of the nucleic acid according to the present invention comprises 17 to 25 base pairs, preferably 19 to 23 base pairs and more preferably 19 to 21 base pairs.

The fourth sub-aspect of the first aspect of the present invention is related to a nucleic acid according to the present invention, whereby the first and/or the second stretch comprise an overhang of 1 to 5 nucleotides at the 3′ end. The design of the nucleic acid in accordance with this sub-aspect is described in WO 02/44321.

In a fifth sub-aspect of the first aspect of the present invention the nucleic acid according to the present invention is a double-stranded nucleic acid which is a chemically synthesized double-stranded short interfering nucleic acid (siNA) molecule which directs cleavage of a CD31 mRNA, preferably via RNA interference, wherein each strand of said siNA molecule is 18 to 27 or 19 to 29 nucleotides in length and said siNa molecule comprises at least one chemically modified nucleotide non-nucleotide. The design of the nucleic acid in accordance with this sub-aspect is described in more detail in international patent application WO03/070910 and UK patent 2 397 062.

In one embodiment thereof the siNA molecule comprises no ribonucleotides. In another embodiment, the siNA molecule comprises one or more nucleotides. In another embodiment chemically modified nucleotide comprises a 2′-deoxy nucleotide. In another embodiment chemically modified nucleotide comprises a 2′-deoxy-2′-fluoro nucleotide. In another embodiment chemically modified nucleotide comprises a 2′-O-methyl nucleotide. In another embodiment chemically modified nucleotide comprises a phosphorothioate internucleotide linkage. In a further embodiment the non-nucleotide comprises an abasic moiety, whereby preferably the abasic moiety comprises an inverted deoxyabasic moiety. In another embodiment non-nucleotide comprises a glyceryl moiety.

In a further embodiment, the first strand and the second strand are connected via a linker molecule. Preferably, the linker molecule is polynucleotide linker. Alternatively, the linker molecule is a non-nucleotide linker.

In a further embodiment of the nucleic acid according to the fifth sub-aspect, the pyrimidine nucleotides in the second strand are 2′-O-methylpyrimidine nucleotides.

In a further embodiment of the nucleic acid according to the fifth sub-aspect, the purine nucleotides in the second strand are 2′-deoxy purine nucleotides.

In a further embodiment of the nucleic acid according to the fifth sub-aspect, the pyrimidine nucleotides in the second strand are 2′-deoxy-2′-fluoro pyrimidine nucleotides.

In a further embodiment of the nucleic acid according to the fifth sub-aspect, the second strand includes a terminal cap moiety at the 5′ end, the 3′ end or both the 5′ end and the 3′ end.

In a further embodiment of the nucleic acid according to the fifth sub-aspect, the pyrimidine nucleotides in the first strand are 2′-deoxy-2′-fluoro pyrimidine nucleotides.

In a further embodiment of the nucleic acid according to the fifth sub-aspect, the purine nucleotides in the first strand are 2′-O-methyl purine nucleotides.

In a further embodiment of the nucleic acid according to the fifth sub-aspect, the purine nucleotides in the first strand are 2′-deoxy purine nucleotides.

In a further embodiment of the nucleic acid according to the fifth sub-aspect, the first strand comprises a phosphorothioate internucleotide linkage at the 3′ end of the first strand.

In a further embodiment of the nucleic acid according to the fifth sub-aspect, the first strand comprises a glyceryl modification at the 3′ end of the first strand.

In a further embodiment of the nucleic acid according to the fifth sub-aspect, about 19 nucleotides of both the first and the second strand are base-paired and wherein preferably at least two 3′ terminal nucleotides of each strand of the siNA molecule are not base-paired to the nucleotides of the other strand. Preferably, each of the two 3′ terminal nucleotides of each strand of the siNA molecule are 2′-deoxy-pyrimidines. More preferably, the 2′deoxy-pyrimidine is 2′ deoxy-thymidine.

In a further aspect of the nucleic acid according to the fifth sub-aspect, the 5′ end of the first strand comprises a phosphate group.

In one embodiment particularly of the fifth sub-aspect of the nucleic acid according to the present invention, a siNA molecule of the invention comprises modified nucleotides while maintaining the ability to mediate RNAi. The modified nucleotides can be used to improve in vitro or in vivo characteristics such as stability, activity, and/or bioavailability. For example, a siNA molecule of the invention can comprise modified nucleotides as a percentage of the total number of nucleotides present in the siNA molecule. As such, a siNA molecule of the invention can generally comprise about 5% to about 100% modified nucleotides (e.g., 5%, 10%, 15%, 20%, 25%, 30%, 35%, 40%, 45%, 50%, 55%, 60%, 65%, 70%, 75%, 80%, 85%, 90%, 95% or 100% modified nucleotides). The actual percentage of modified nucleotides present in a given siNA molecule will depend on the total number of nucleotides present in the siNA. If the siNA molecule is single-stranded, the percent modification can be based upon the total number of nucleotides present in the single-stranded siNA molecules. Likewise, if the siNA molecule is double-stranded, the percent modification can be based upon the total number of nucleotides present in the sense strand, antisense strand, or both the sense and antisense strands.

In a non-limiting example, the introduction of chemically-modified nucleotides into nucleic acid molecules particularly of the fifth sub-aspect of the nucleic acid according to the present invention provides a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example, when compared to an all-RNA nucleic acid molecule, the overall activity of the modified nucleic acid molecule can be greater than that of the native molecule due to improved stability and/or delivery of the molecule. Unlike native unmodified siNA, chemically-modified siNA can also minimize the possibility of activating interferon activity in humans.

Preferably in connection with the fifth sub-aspect of the nucleic acid according to the present invention, the antisense strand, i.e. the first strand, of a siNA molecule of the invention can comprise a phosphorothioate internucleotide linkage at the 3′-end of said antisens region. The antisense strand can comprise about one to about five phosphorothioate internucleotide linkages at the 5′-end of said antisense region. The 3′-terminal nucleotide overhangs of a siNA molecule of the invention can comprise ribonucleotides or deoxyribonucleotides that are chemically-modified at a nucleic acid sugar, base or backbone. The 3′-terminal nucleotide overhangs can comprise one or more universal base ribonucleotides. The 3′-terminal nucleotide overhangs can comprise one or more acyclic nucleotides.

It will be acknowledged by the ones skilled in the art that particularly the embodiment of the present invention which comprises a loop made of nucleotides is suitable to be used and expressed by a vector. Preferably, the vector is an expression vector. Such expression vector is particular useful in any gene therapy approach. Accordingly, such vector can be used for the manufacture of a medicament which is preferable to be used for the treatment of the diseases disclosed herein. It will, however, also be acknowledged by the ones skilled in the art that any embodiment of the nucleic acid according to the present invention which comprises any non-naturally occurring modification cannot immediately be used for expression in a vector and an expression system for such vector such as a cell, tissue, organ and organism. However, it is within the present invention that the modification may be added to or introduced into the vector derived or vector expressed nucleic acid according to the present invention, after the expression of the nucleic assay by the vector. A particularly preferred vector is a plasmid vector or a viral vector. The technical teaching on how to use siRNA molecules and RNAi molecules in an expression vector is, e.g., described in international patent application WO 01/70949. It will be acknowledged by the ones skilled in the art that such vector is preferably useful in any method either therapeutic or diagnostic where a sustained presence of the nucleic acid according to the present invention is desired and useful, respectively, whereas the non-vector nucleic acid according to the present invention and in particular the chemically modified or chemically synthesized nucleic acid according to the present invention is particularly useful where the transient presence of the molecule is desired or useful.

Methods for the synthesis of the nucleic acid molecule described herein are known to the ones skilled in the art. Such methods are, among others, described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference.

In a further aspect the present invention is related to lipoplexes comprising the nucleic acid according to the present invention. Such lipoplexes consist of one or several nucleic acid molecules and one or several liposomes. In a preferred embodiment a lipoplex consists of one liposome and several nucleic acid molecules.

The lipoplex can be charaterised as follows. The lipoplex according to the present invention has a zeta-potential of about 40 to 55 mV, preferably about 45 to 50 mV. The size of the lipoplex according to the present invention is about 80 to 200 nm, preferably of about 100 to 140 nm, and more preferably of about 110 nm to 130 mm, as determined by dynamic light scattering (QELS) such as, e.g., by using an N5 submicron particle size analyzer from Beckman Coulter according to the manufacturer's recommendation.

The liposome as forming part of the lipoplex according to the present invention is preferably a positively charged liposome consisting of

a) about 50 mol % β-arginyl-2,3-diaminopropionic acid-N-palmityl-N-oleyl-amide trihydrochloride, preferably β-(L-arginyl)-2,3-L-diaminopropionic acid-N-palmityl-N-oleyl-amide tri-hydrochloride,
b) about 48 to 49 mol % 1,2-diphytanoyl-sn-glycero-3-phosphoethanolamine (DPhyPE), and
c) about 1 to 2 mol % 1,2-distearoyl-sn-glycero-3-phosphoethanolamine-polyethylen-glycole, preferably N—(Carbonyl-methoxypolyethyleneglycol-2000)-1,2-distearoyl-sn-glycero-3-phosphoethanolamine sodium salt.

The lipoplex and lipid composition forming the liposomes is preferably contained in a carrier. However, the lipoplex can also be present in a lyophilised form. The lipid composition contained in a carrier usually forms a dispersion. More preferably, the carrier is an aqueous medium or aqueous solution as also further characterised herein. The lipid composition typically forms a liposome in the carrier, whereby such liposome preferably also contains the carrier inside.

The lipid composition contained in the carrier and the carrier, respectively, preferably has an osmolarity of about 50 to 600 mosmole/kg, preferably about 250-350 mosmole/kg, and more preferably about 280 to 320 mosmole/kg.

The liposomes preferably formed by the first lipid component and optionally also by the first helper lipid, preferably in combination with the first lipid component, preferably exhibit a particle size of about 20 to 200 nm, preferably about 30 to 100 nm, and more preferably about 40 to 80 nm.

Furthermore, it will be acknowledged that the size of the particles follows a certain statistical distribution.

A further optional feature of the lipid composition in accordance with the present invention is that the pH of the carrier is preferably from about 4.0 to 6.0. However, also other pH ranges such as from 4.5 to 8.0, preferably from about 5.5 to 7.5 and more preferably about 6.0 to 7.0 are within the present invention.

For realizing these particular features various measures may be taken. For adjusting the osmolarity, for example, a sugar or a combination of sugars is particularly useful. Insofar, the lipid composition of the present invention may comprise one or several of the following sugars: sucrose, trehalose, glucose, galactose, mannose, maltose, lactulose, inulin and raffinose, whereby sucrose, trehalose, inulin and raffinose are particularly preferred. In a particularly preferred embodiment the osmolarity mostly adjusted by the addition of sugar is about 300 mosmole/kg which corresponds to a sucrose solution of 270 mM or a glucose solution of 280 mM. Preferably the carrier is isotonic to the body fluid into which such lipid composition is to be administered. As used herein the term that the osmolarity is mostly adjusted by the addition of sugar means that at least about 80%, preferably at least about 90% of the osmolarity is provided by said sugar or a combination of said sugars.

If the pH of the lipid composition of the present invention is adjusted, this is done by using buffer substances which, as such, are basically known to the one skilled in the art. Preferably, basic substances are used which are suitable to compensate for the basic characteristics of the cationic lipids and more specifically of the ammonium group of the cationic head group. When adding basic substances such as basic amino acids and weak bases, respectively, the above osmolarity is to be taken into consideration. The particle size of such lipid composition and the liposomes formed by such lipid composition is preferably determined by dynamic light scattering such as by using an N5 submicron particle size analyzer from Beckman Coulter according to the manufacturer's recommendation.

If the lipid composition contains one or several nucleic acid(s), such lipid composition usually forms a lipoplex complex, i.e. a complex consisting of a liposome and a nucleic acid. The more preferred concentration of the overall lipid content in the lipoplex in preferably isotonic 270 mM sucrose or 280 mM glucose is from about 0.01 to 100 mg/ml, preferably 0.01 to 40 mg/ml and more preferably 0.01 to 25 mg/ml. It is to be acknowledged that this concentration can be increased so as to prepare a reasonable stock, typically by a factor of 2 to 3. It is also within the present invention that based on this, a dilution is prepared, whereby such dilution is typically made such that the osmolarity is within the range specified above. More preferably, the dilution is prepared in a carrier which is identical or in terms of function and more specifically osmolarity similar to the carrier used in connection with the lipid composition or in which the lipid composition is contained. In the embodiment of the lipid composition of the present invention whereby the lipid composition also comprises a nucleic acid, preferably a functional nucleic acid such as, but not limited to, a siRNA, the concentration of the functional nucleic acid, preferably of siRNA in the lipid composition is about 0.2 to 0.4 mg/ml, preferably 0.28 mg/ml, and the total lipid concentration is about 1.5 to 2.7 mg/ml, preferably 2.17 mg/ml. It is to be acknowledged that this mass ratio between the nucleic acid fraction and the lipid fraction is particularly preferred, also with regard to the charge ratio thus realized. In connection with any further concentration or dilution of the lipid composition of the present invention, it is preferred that the mass ratio and the charge ratio, respectively, realized in this particular embodiment is preferably maintained despite such concentration or dilution.

Such concentration as used in, for example, a pharmaceutical composition, can be either obtained by dispersing the lipid in a suitable amount of medium, preferably a physiologically acceptable buffer or any carrier described herein, or can be concentrated by appropriate means. Such appropriate means are, for example, ultra filtration methods including cross-flow ultra-filtration. The filter membrane may exhibit a pore width of 1.000 to 300.000 Da molecular weight cut-off (MWCO) or 5 nm to 1 μm. Particularly preferred is a pore width of about 10.000 to 100.000 Da MWCO. It will also be acknowledged by the one skilled in the art that the lipid composition more specifically the lipoplexes in accordance with the present invention may be present in a lyophilized form. Such lyophilized form is typically suitable to increase the shelve life of a lipoplex. The sugar added, among others, to provide for the appropriate osmolarity, is used in connection therewith as a cryo-protectant. In connection therewith it is to be acknowledged that the aforementioned characteristics of osmolarity, pH as well as lipoplex concentration refers to the dissolved, suspended or dispersed form of the lipid composition in a carrier, whereby such carrier is in principle any carrier described herein and typically an aqueous carrier such as water or a physiologically acceptable buffer, preferably an isotonic buffer or isotonic solution.

Apart from these particular formulations, the nucleic acid molecules according to the present invention may also be formulated in pharmaceutical compositions as known in the art.

Accordingly, the nucleic acid molecules according to the present invention can preferably be adapted for use as medicaments and diagnostics, alone or in combination with other therapies. For example, a nucleic acid molecule according to the present invention can comprise a delivery vehicle, including liposomes, for administration to a subject, carriers and diluents and their salts, and/or can be present in pharmaceutically acceptable formulations. Methods for the delivery of nuclecic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Memb. Biol., 16, 129-140; Hofland and Huang, 1999, Handb. Exp. Pharmacol., 137, 165-192; and Lee et al., 2000, ACS Symp. Ser., 752, 184-192 all of which are incorporated herein by reference. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not limited to, encapsulation in liposomes, by iontophoresis, or by incorporation into other vehicles, such as hydrogels, cyclodextrins (see for example Gonzalez et al., 1999, Bioconjugate Chem., 10, 1068-1074), biodegradable nanocapsules, and bioadhesive microspheres, or by proteinaccous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262. The molecules of the instant invention can be used as pharmaceutical agents. Preferably, pharmaceutical agents prevent, modulate the occurrence, or treat (alleviate a symptom to some extent, preferably all of the symptoms) of a disease state in a subject.

Thus, there is provided a pharmaceutical composition comprising one or more nucleic acid(s) according to the present invention in an acceptable carrier, such as a stabilizer, buffer, and the like. The polynucleotide(s) or nucleic acid(s) of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions, suspensions for injectable administration, and the other compositions known in the art.

There are further provided pharmaceutically acceptable formulations of the nucleic acid molecules according to the present invention. These formulations include salts of the above compounds, e.g., acid addition salts, for example, salts of hydrochloric, hydrobromic, acetic acid, and benzene sulfonic acid.

A pharmacological composition or formulation preferably refers to a composition or formulation in a form suitable for administration, e.g., systemic administration, into a cell or subject, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes that lead to systemic absorption include, without limitation: intravenous, subcutaneous, intraperitoneal, inhalation, oral, intrapulmonary and intramuscular. Each of these administration routes exposes the siNA molecules siRNA molecules of the invention to an accessible diseased tissue. The rate of entry of a drug, such as the nucleic acid molecules of the present invention, into the circulation has been shown to be a function of molecular weight or size. The use of a liposome or other drug carrier comprising the nucleic acid(s) according to the present invention can potentially localize the drug, for example, in certain tissue types, such as neoplastic tissue(s). A liposome formulation that can facilitate the association of drug with the surface of cells, such as lymphocytes and macrophages is also useful. This approach can provide enhanced delivery of the drug to target cells by taking advantage of the specificity of macrophage and lymphocyte immune recognition of abnormal cells, such as cells forming the neoplastic tissue.

By “pharmaceutically acceptable formulation” is preferably meant a composition or formulation that allows for the effective distribution of the nucleic acid molecules according to the present invention in the physical location most suitable for their desired activity. Non-limiting examples for agents suitable for formulation with the nucleic acid molecules according to the present invention include: P-glycoprotein inhibitors (such as Pluronic P85), which can enhance entry of drugs into the CNS (Jollict-Riant and Tillement, 1999, Fundam. Clin. Pharmacol., 13, 16-26); biodegradable polymers, such as poly (DL-lactide-co-glycolide) microspheres for sustained release delivery after intracerebral implantation (Emerich, D F et al., 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the present invention include material described in Boado et al., 1998, J. Pharm. Sci., 87, 1308-1315; Tyler et al., 1999, FEBS Lett., 421, 280-284; pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

There is also provided the use of a composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24780; Choi et al., Internaional PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

There are moreover provided herein compositions prepared for storage of administration that include a pharmaceutically effective amount of the desired compounds such as the nucleic acid molecules according to the present invention, in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or threat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules according to the present invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrahecal injection or infusion techniques and the like. In addition, there is provided a pharmaceutical formulation comprising a nucleic acid molecule of the invention and a pharmaceutically acceptable carrier. One or more nucleic acid molecules according to the present invention can be present in association with one or more non-toxic pharmaceutically acceptable carriers and/or diluents and/or adjuvants, and if desired other active ingredients. The pharmaceutical compositions containing nucleic acid molecules according to the present invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the person skilled in the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials such as the nucleic acid(s) according to the present invention in a mixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents can be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxyoctanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavouring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavouring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavouring and coloring agents, can also be present.

Pharmaceutical compositions of the invention can also be in the form of oil-in-water emulsions. The oily phase can be a vegetable oil or a mineral oil or mixture of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavouring agents.

Syrups and elixirs can be formulated with sweetening agents, for example glycerol, propylene glycol, sorbitol, glucose or sucrose. Such formulations can also contain a demulcent, a preservative and flavouring and coloring agents. The pharmaceutical compositions can be in the from of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parenterally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels for the medicament and pharmaceutical composition, respectively, can be determined by those skilled in the art by routine experimentation.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

For administration of the medicament according to the present invention to non-human animals such as dogs, cats, horses, cattle, pig, goat, sheep, mouse, rat, hamster and guinea pig, the composition can preferably also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

In one embodiment, there are provided compositions suitable for administering the nucleic acid molecules according to the present invention to specific cell types, whereby such compositions typically incorporate one or several of the following principles and molecules, respectively. For example, the asialoglycoprotein receptor (ASGPr) (Wu and Wu, 1987, J. Biol. Chem. 262, 4429-4432) is unique to hepatocytes and binds branched galactose-terminal glycoproteins, such as asialoorosomucoid (ASOR). In another example, the folate receptor is overexpressed in many cancer cells. Binding of such glycoproteins, synthetic glycoconjugates, or folates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611-620; Connolly et al., 1982, J. Biol. Chem., 257, 939-945). Lee and Lee, 1987. Glycoconjugate J, 4, 317-328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388-1395). The use of galactose, galactosamine, or folate based conjugates to transport exogenous compounds across cell membranes can provide a targeted delivery approach to, for example, the treatment of liver disease, cancers of the liver, or other cancers. The use of bioconjugates can also provide a reduction in the required dose of therapeutic compounds required for treatment. Furthermore, therapeutic bioavailability, pharmacodynamics, and pharmacokinetic parameters can be modulated through the use of nucleic acid bioconjugates of the invention. Non-limiting examples of such bioconjugates are described in Vargeese et al., U.S. Ser. No. 10/201,394, filed Aug. 13, 2001; and Matulic-Adamic et al., U.S. Ser. No. 60/362,016, filed Mar. 6, 2002.

The nucleic acid molecules, in their various embodiments, according to the present invention, the vector, cell, medicament, composition and in particular pharmaceutical composition containing the same, tissue and animal, respectively, according to the present invention containing such (a) nucleic acid molecule(s) can be used in both for therapeutic use as well as in the diagnostic and research field.

Due to the distribution of CD1 in various tissues and vascular endothelium involved in the following diseases, the nucleic acid molecule(s) according to the present invention may be used for the treatment and/or prevention of said diseases.

Accordingly, the nucleic acid molecules as disclosed herein and the medicaments and pharmaceutical compositions containing the same may be used for both pro- and anti-angiogenic therapies including diseases characterized or caused by insufficient, abnormal or excessive angiogenesis. Such diseases comprise infectious diseases, autoimmune disorders, vascular malformation, atherosclerosis, transplant arteriopathy, obesity, psoriasis, warts, allergic dermatitis, persistent hyperplastic vitrous syndrome, diabetic retinopathy, retinopathy of prematurity, age-related macular disease, choroidal neovascularization, primary pulmonary hypertension, asthma, nasal polyps, inflammatory bowel and periodontal disease, ascites, peritoneal adhesions, endometriosis, uterine bleeding, ovarian cysts, ovarian cancer, ovarian hyperstimulation, arthritis, synovitis, osteomyelitis, osteophyte formation and stroke, ulcers, atherosclerosis and rheumatoid arthritis.

Further diseases are those involving or characterized by a neoplastic tissue. As preferably used herein, the term neoplastic tissues refers to tissues which are generated by an organism, tissue or cells of such organism which are not intended to be generated and which are deemed as pathologic, i.e. not present in a subject not suffering from such a respective disease. Also, as preferably used herein, a neoplastic disease is any disease which, either directly or indirectly, arises from the presence of a neoplastic tissue, whereby preferably such neoplastic tissue arises from the dysregulated or uncontrolled, preferably autonomous growth of a/the tissue. The term neoplastic diseases preferably also comprises benign as well as malignant neoplastic diseases. More preferably, the neoplastic diseases are selected from the group comprising any cancer of, e.g., bone, breast, prostate, digestive system, colorectal, liver, lung, kideney, urogenital, pancreatic, pituitary, testicular, orbital, head and neck, central nervous system, and respiratory organs.

Further specific diseases which, in principle, can be treated using the pharmaceutical composition and the medicament in accordance with the present invention, comprising such lipid composition and lipoplex according to the present invention, respectively, may be taken from the following list: Acute Lymphoblastic Leukemia (Adult), Acute Lymphoblastic Leukemia (Childhood), Acute Myeloid Leukemia (Adult), Acute Myeloid Leukemia (Childhood), Adrenocortical Carcinoma, Adrenocortical Carcinoma (Childhood), AIDS-Related Cancers, AIDS-Related Lymphoma, Anal Cancer, Astrocytoma (Childhood), Cerebellar Astrocytoma (Childhood) Cerebral, Bile Duct Cancer, Extrahepatic, Bladder Cancer, Bladder Cancer (Childhood), Bone Cancer, Osteosarcoma/Malignant Fibrous Histiocytoma, Brain Stem Glioma (Childhood), Brain Tumor (Adult), Brain Tumor, Brain Stem Glioma (Childhood), Brain Tumor, Cerebellar Astrocytoma, (Childhood), Brain Tumor, Cerebral Astrocytoma/Malignant Glioma, (Childhood), Brain Tumor, Ependymoma, (Childhood), Brain Tumor, Medulloblastoma, (Childhood), Brain Tumor, Supratentorial Primitive Neuroectodermal Tumors (Childhood), Brain Tumor, Visual Pathway and Hypothalamic Glioma (Childhood), Brain Tumor (Childhood), Breast Cancer, Breast Cancer, (Childhood), Breast Cancer, Male, Bronchial Adenomas/Carcinoids (Childhood), Burkitt's Lymphoma, Carcinoid Tumor (Childhood), Carcinoid Tumor, Gastrointestinal, Carcinoma of Unknown Primary, Central Nervous System Lymphoma, Primary, Cerebellar Astrocytoma (Childhood), Cerebral Astrocytoma/Malignant Glioma (Childhood), Cervical Cancer, Chronic Lymphocytic Leukemia, Chronic Myelogenous Leukemia, Chronic Myeloproliferative Disorders, Colon Cancer, Colorectal Cancer (Childhood), Cutaneous T-Cell Lymphoma, Endometrial Cancer, Ependymoma (Childhood), Esophageal Cancer, Esophageal Cancer (Childhood), Ewing's Family of Tumors, Extracranial Germ Cell Tumor (Childhood), Extragonadal Germ Cell Tumor, Extrahepatic Bile Duct Cancer, Eye Cancer, Intraocular Melanoma, Eye Cancer, Retinoblastoma, Gallbladder Cancer, Gastric (Stomach) Cancer, Gastric (Stomach) Cancer (Childhood), Gastrointestinal Carcinoid Tumor, Germ Cell Tumor, Extracranial (Childhood), Germ Cell Tumor, Extragonadal, Germ Cell Tumor, Ovarian, Gestational Trophoblastic Tumor, Glioma (Adult), Glioma (Childhood) Brain Stem, Glioma (Childhood) Cerebral Astrocytoma, Glioma (Childhood) Visual Pathway and Hypothalamic, Hairy Cell Leukemia, Head and Neck Cancer, Hepatocellular (Liver) Cancer (Adult) (Primary), Hepatocellular (Liver) Cancer (Childhood) (Primary), Hodgkin's Lymphoma (Adult), Hodgkin's Lymphoma (Childhood), Hypopharyngeal Cancer, Hypothalamic and Visual Pathway Glioma (Childhood), Intraocular Melanoma, Islet Cell Carcinoma (Endocrine Pancreas), Kaposi's Sarcoma, Kidney (Renal Cell) Cancer, Kidney Cancer (Childhood), Laryngeal Cancer, Laryngeal Cancer, (Childhood), Leukemia, Acute Lymphoblastic, (Adult), Leukemia, Acute Lymphoblastic (Childhood), Leukemia, Acute Myeloid (Adult), Leukemia, Acute Myeloid (Childhood), Leukemia, Chronic Lymphocytic Leukemia, Chronic Myelogenous, Leukemia, Hairy Cell, Lip and Oral Cavity Cancer, Liver Cancer (Adult) (Primary), Liver Cancer (Childhood) (Primary), Lung Cancer, Non-Small Cell, Lung Cancer, Small Cell, Lymphoma, AIDS-Related, Lymphoma, Burkitt's, Lymphoma, Cutaneous T-Cell, Lymphoma, Hodgkin's (Adult), Lymphoma, Hodgkin's (Childhood), Lymphoma, Non-Hodgkin's (Adult), Lymphoma, Non-Hodgkin's (Childhood), Lymphoma, Primary Central Nervous System, Macroglobulinemia, Waldenstrom's Malignant Fibrous Histiocytoma of Bone/Osteosarcoma, Medulloblastoma (Childhood), Melanoma, Melanoma, Intraocular (Eye), Merkel Cell Carcinoma, Mesothelioma (Adult) Malignant, Mesothelioma (Childhood), Metastatic Squamous Neck Cancer with Occult Primary, Multiple Endocrine Neoplasia Syndrome (Childhood), Multiple Myeloma/Plasma Cell Neoplasm, Mycosis Fungoides, Myelodysplastic Syndromes, Myelodysplastic/Myeloproliferative Diseases, Myelogenous Leukemia, Chronic, Myeloid Leukemia (Adult) Acute, Myeloid Leukemia (Childhood) Acute, Myeloma, Multiple, Myeloproliferative Disorders, Chronic, Nasal Cavity and Paranasal Sinus Cancer, Nasopharyngeal Cancer, Nasopharyngeal Cancer (Childhood), Neuroblastoma, Non-Hodgkin's Lymphoma (Adult), Non-Hodgkin's Lymphoma (Childhood), Non-Small Cell Lung Cancer, Oral Cancer (Childhood), Oral Cavity Cancer, Lip and Oropharyngeal Cancer, Osteosarcoma/Malignant Fibrous Histiocytoma of Bone, Ovarian Cancer (Childhood), Ovarian Epithelial Cancer, Ovarian Germ Cell Tumor, Ovarian Low Malignant Potential Tumor, Pancreatic Cancer, Pancreatic Cancer (Childhood), Pancreatic Cancer, Islet Cell, Paranasal Sinus and Nasal Cavity Cancer, Parathyroid Cancer, Penile Cancer, Pheochromocytoma, Pineoblastoma and Supratentorial Primitive Neuroectodermal Tumors (Childhood), Pituitary Tumor, Plasma Cell Neoplasm/Multiple Myeloma, Pleuropulmonary Blastoma, Pregnancy and Breast Cancer, Pregnancy and Hodgkin's Lymphoma, Pregnancy and Non-Hodgkin's Lymphoma, Primary Central Nervous System Lymphoma, Prostate Cancer, Rectal Cancer, Renal Cell (Kidney) Cancer, Renal Cell (Kidney) Cancer (Childhood), Renal Pelvis and Ureter, Transitional Cell Cancer, Retinoblastoma, Rhabdomyosarcoma (Childhood), Salivary Gland Cancer, Salivary Gland Cancer (Childhood), Sarcoma, Ewing's, Sarcoma, Kaposi's, Sarcoma, Soft Tissue, (Adult), Sarcoma, Soft Tissue, (Childhood), Sarcoma, Uterine, Sezary Syndrome, Skin Cancer (non-Melanoma), Skin Cancer, (Childhood), Skin Cancer (Melanoma), Skin Carcinoma, Merkel Cell, Small Cell Lung Cancer, Small Intestine Cancer, Soft Tissue Sarcoma (Adult), Soft Tissue Sarcoma (Childhood), Squamous Cell Carcinoma, Squamous Neck Cancer with Occult Primary, Metastatic Stomach (Gastric) Cancer, Stomach (Gastric) Cancer (Childhood), Supratentorial Primitive Neuroectodermal Tumors (Childhood), T-Cell Lymphoma, Cutaneous, Testicular Cancer, Thymom (Childhood), Thymoma and Thymic Carcinoma, Thyroid Cancer Thyroid Cancer (Childhood), Transitional Cell Cancer of the Renal Pelvis and Ureter, Trophoblastic Tumor, Gestational, Ureter and Renal Pelvis, Transitional Cell Cancer, Urethral Cancer, Uterine Cancer, Endometrial, Uterine Sarcoma, Vaginal Cancer, Visual Pathway and Hypothalamic Glioma (Childhood), Vulvar Cancer, Waldenstrom's Macroglobulinemia, Wilms' Tumor.

It is to be acknowledged that the various diseases described herein for the treatment and prevention of which the pharmaceutical composition according to the present invention may be used, are also those diseases for the prevention and/or treatment of which the medicament described herein can be used, and vice versa.

As used herein the term treatment of a disease shall also comprise prevention of such disease.

Further features, embodiments and advantages may be taken from the following figures, whereby

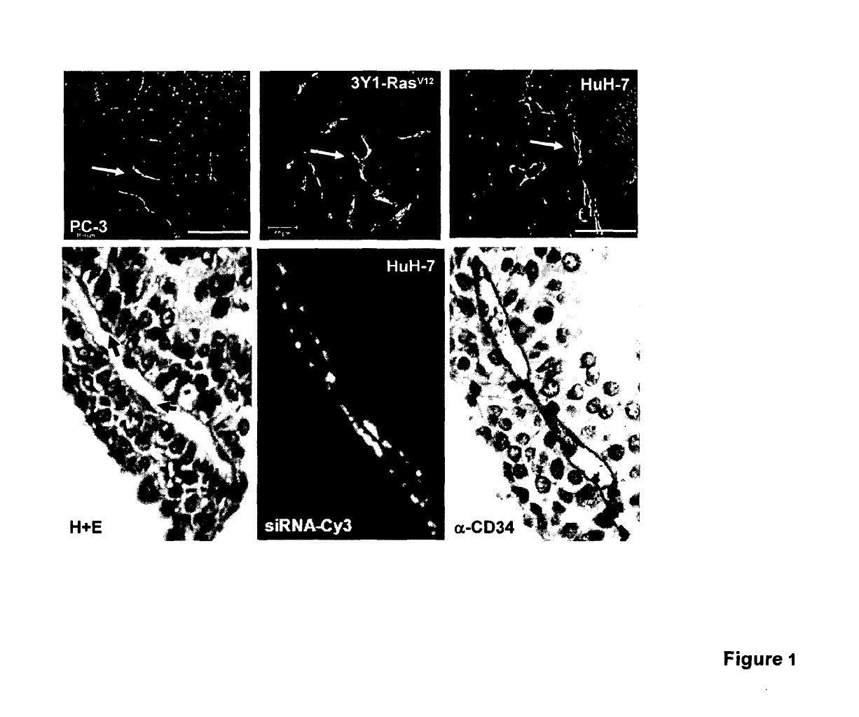

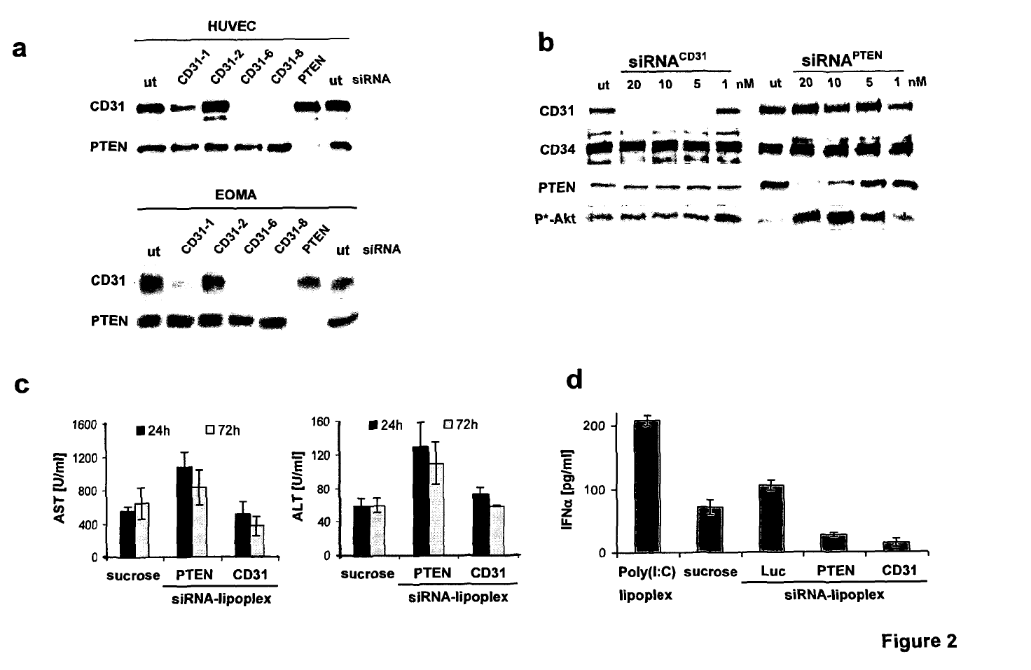

FIG. 1 show confocal microscopic pictures of endothelial cells of different established tumors;

FIG. 2a, b show the result of a Western blot analysis of a knockdown experiment using different CD31 specific siRNA molecules (a) and different amounts of a distinct CD31 specific siRNA molecule (b);

FIG. 2c shows a diagram indicating the activity of various liver enzymes upon administration of various siRNA molecules;

FIG. 2d shows a diagram indicating IFN-alpha response upon systemic administration of different lipoplexes;

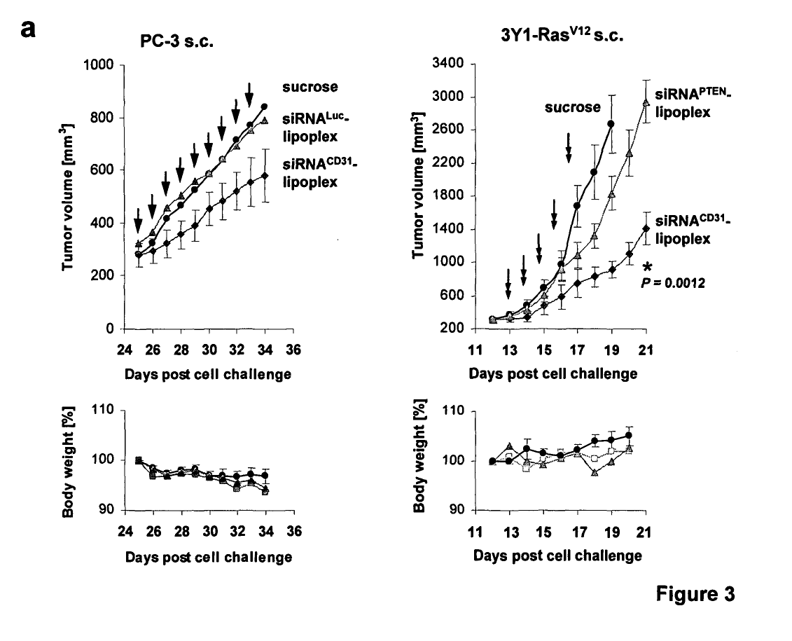

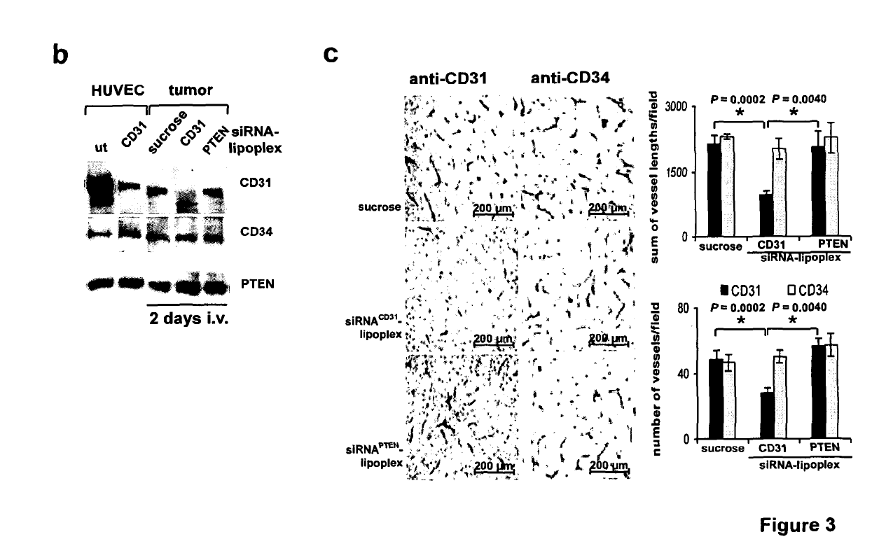

FIG. 3a shows diagrams illustrating the effect of different agents on the volume of two different tumors and body weight, respectively, as a function of days post cell challenge;

FIG. 3b shows the result of a Western blot analysis of a knockdown experiment in tumor bearing mice using different lipoplexes;

FIG. 3c shows the result of immunostaining using anti-CD31 antibodies in tumor sections of mice treated with different lipoplexes (left panel) and diagrams indicating the sum of vessels and number of vessels each per field upon treatment with different lipoplexes;

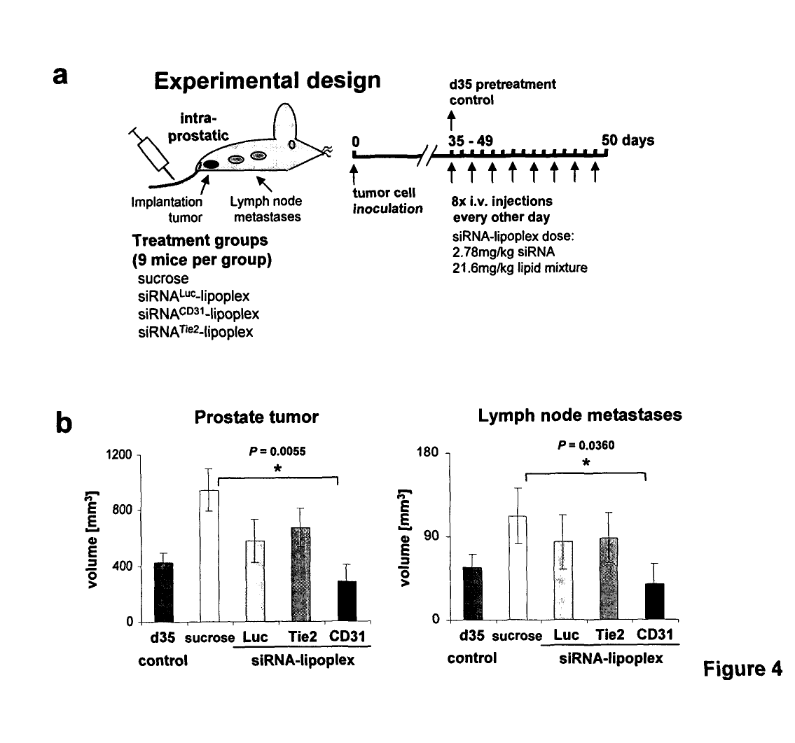

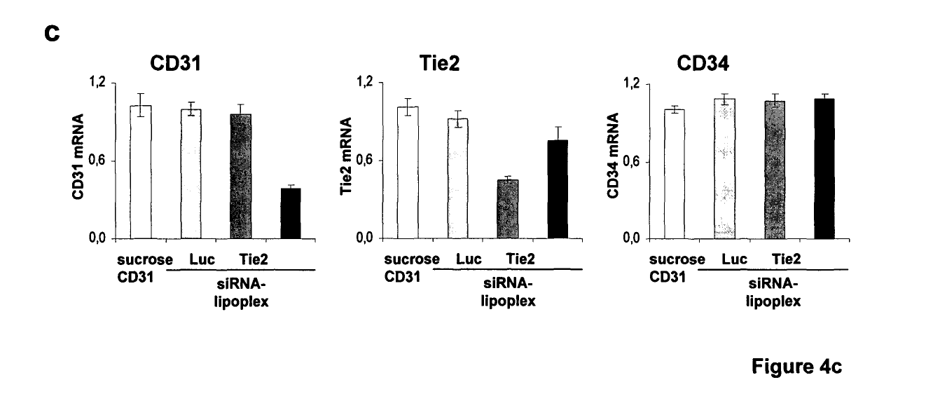

FIG. 4a shows a schematic of the experimental design;

FIG. 4b shows the volume of prostate tumor and lymph node metastases, respectively upon treatment with different lipoplexes;

FIG. 4c shows mRNA knockdown in lung tissue upon treatment with different lipoplexes;

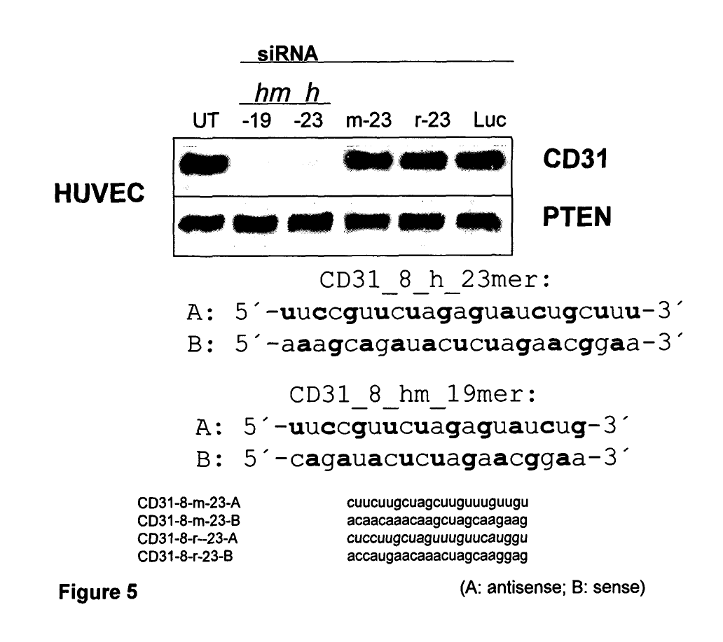

FIG. 5 shows the result of a Western Blot analysis using either a target specific 23mer siRNA or a target specific 19mer siRNA for knock down of the target.

### EXAMPLE 1

### Materials and Methods

### Antibodies

The following antibodies were used in this study: rabbit anti-PTEN (Ab-2, Neomarkers), goat anti-CD31 and rabbit anti-CD34 (Santa Cruz Biotechnology), rabbit anti-phosphorylated Akt (S473) (Cell Signaling Technology), the immunohistochemistry-specific rabbit anti-phosphorylated-Akt (S473) (Cell Signaling Technology)), anti-CD31/PECAM-1 (Santa Cruz Biotechnology) (alternatively for cryosections rat CD31, Pharmingen), and rat-monoclonal anti-CD34 (Cedarlane goat polyclonal).

### Cell Lines

PC-3 cell line was obtained from American Type Culture Collection and cultivated according to the ATCC's recommendation. Human hepatoma cell line HuH-7 was available at MDC, Berlin. Rat 3Y1 cells expressing oncogenic Ras^{V12} were generated by transduction of inducible Ras^{V12} as described (Leenders et al., 2004). Transfections and proteins extracts for immunoblotting were carried out as previously described (Santel et al., 2006).

Delivery of siRNA-Cy3 Lipoplexes in Tumor Bearing Mice

In vivo delivery experiments using fluorescently labeled siRNA-Cy3 lipoplexes were performed by administering siRNA lipoplexes intravenously through single tail vein injection of 200 μl solution at a final dose of 1.88 mg/kg siRNA-Cy3 and 14.5 mg/kg lipid. Mice were sacrificed 4 hours post injection and fluorescence uptake examined by microscopy on formalin fixed, paraffin embedded tissue sections.

### Histological Analysis and Microscopy

Immunofluorescence analysis on culture cells was carried out as described (Santel et al., 2006). Tissues were instantly fixed in 4.5% buffered formalin for 16 hours and processed for paraffin sectioning by standard protocols. Tissue sections were stained with anti-CD31 or anti-CD34 to visualize endothelial cells in paraffin sections. Immunohistochemistry with hematoxylin counterstaining as well as hematoxylin/eosin staining (H+E) was performed according to standard protocols. For in vivo uptake studies of fluorescently labeled siRNAs, paraffin sections were deparaffinizied, counterstain end with Sytox Green dye (Molecular Probes 100 nM) and examined by epifluorescence (Zeiss Axioplan microscope) or confocal (Zeiss LSM510 Meta) microscopy.

### Determination of Microvessel Density (MVD)

The number of microvessels was determined by counting CD31-/CD34-positive vessels in 3-8 randomly selected areas of single tumor sections (Fox and Harris, 2004). Vessel number as vascular units was evaluated regardless of shape, branch points and size lumens (referring to “number of vessels”). Additionally, vascular density was assessed by determination of total length of CD31-/CD34-positive vessel structures (referring to “sum of vessel lengths”) using the Axiovision 3.0 software (Zeiss). Counting was performed by scanning tumor sections at 200× magnification with a Zeiss Axioplan light microscope.

### Tumor Xenograft Experiments

Male Hsd:NMRI-nu/nu mice (8 weeks old) were used in this study. For tumor therapy experiments on established tumor xenografts, a total of 5.0×10⁶ tumor cells/100 μl PBS (3Y1-Ras^{V12} in the presence of 50% Matrigel) were implanted subcutaneously. Tumor volume was determined using a caliper and calculated according to the formula volume=(length×width²)/2. For tumor therapy experiments siRNA-lipoplex solution was administered i.v. by low pressure, low volume tail vein injection. Established 3Y1-Ras^{V12} tumor mice received a bidaily 200 μl injection for a 30 g mouse (single dose 1.88 mg/kg siRNA and 14.5 mg/kg lipid). In the orthotopic tumor model 2.0×10⁶ PC-3 cells/30 μl PBS were injected into the left dorsolateral lobe of the prostate gland under total body anesthesia (Stephenson et al., 1992). A 30 g mouse with an established prostate tumor received a 300 μl injection of the stock solution mentioned above (equivalent to a dose of 2.17 mg/kg siRNA and 21.6 mg/kg lipid). Animals were killed 50 days post-operation and volumes of tumors (prostate gland) and regional metastases (caudal, lumbar and renal lymph node metastases) were determined as mentioned above. For intrahepatic applications 2.0×10⁶ cells/20 μl PBS were applied by direct injection into the left lateral lobe of the liver. All animal experiments in this study were performed according to approved protocols and in compliance with the guidelines of the Landesamt für Arbeits-, Gesundheitsschutz und technische Sicherheit Berlin, Germany (No. G0264/99).

### Statistical Analysis

Data are expressed as means±s.e.m. Statistical significance of differences was determined by the Mann-Whitney U test. P values <0.05 were considered statistically significant.

### EXAMPLE 2

### CD31 siRNA Molecules

The used siRNA molecules (AtuRNAi, see Table 1.) used in this study are described in (Czauderna et al., 2003a) and were synthesized by BioSpring (Frankfurt a. M., Germany).

TABLE 1

| | |
|---|---|
| siRNA name | sequence 5′ to 3′ |

| | |
|---|---|
| PTEN s | ccaccacagcuagaacuua |
| PTEN as | uaaguucuagcuguggugg |
| PTEN s (control) | ccaccacagcuagaacuua |
| PTEN as (control) | uaaguucuagcuguggugg |
| PTEN s | ccaccacagcuagaacuua |
| PTEN as-Cy3 | uaaguucuagcuguggugg-Cy3 |
| CD31-1 s | ccaacuucaccauccagaa |
| CD31-1 as | uucuggauggugaaguugg |
| CD31-2 s | ggugauagccccgguggau |
| CD31-2 as | auccaccggggcuaucacc |
| CD31-6 s | ccacuucugaacuccaaca |
| CD31-6 as | uguuggaguucagaagugg |
| CD31-8 s | cagauacucuagaacggaa |
| CD31-8 as | uuccguucuagaguaucug |
| Luciferase s | ucgaaguauuccgcguacg |
| Luciferase as | cguacgcggaauacuucga |
| Tie2 s | auaucugggcaaaugaugg |
| Tie2 as | ccaucauuugcccagauau |

Nucleotides with 2′-O-methyl modifications are underlined
S stands for the sense strand which is also referred to herein as the first strand; and As stands for the antisense strand which is also referred to herein as the second strand.

The duplexes formed by CD31-8 as and CD31-8 s, formed by CD31-6 as and CD31-6 s, formed by CD31-1 as and CD31-1 s lack 3′-overhangs, which are chemically stabilized by alternating 2′-O-methyl sugar modifications on both strands, whereby unmodified nucleotides face modified ones on the opposite strand (Table 1) (Czauderna et al., 2003a). These duplexes are also referred to herein as CD31-8, CD31-6 and CD31-1, all of which are particularly preferred embodiments of the nucleic acid molecules in accordance with the present invention.

### EXAMPLE 3

### Lipoplex formulation of CD31 specific siRNA molecules

The novel cationic lipid AtuFECT01 (β-L-arginyl-2,3-L-diaminopropionic acid-N-palmityl-N-oleyl-amide trihydrochloride, Atugen AG), the neutral phospholipid 1,2-diphytanoyl-sn-glycero-3-phosphoethanolamine (DPhyPE) (Avanti Polar Lipids Inc., Alabaster, Ala.) and the PEGylated lipid N-(Carbonyl-methoxypolyethyleneglycol-2000)-1,2-distearoyl-sn-glycero-3-phospho-ethanolamine sodium salt (DSPE-PEG) (Lipoid GmbH, Ludwigshafen, Germany) were mixed in a molar ratio of 50/49/1 by lipid film re-hydration in 300 mM sterile RNase-free sucrose solution to a total lipid concentration of 4.34 mg/ml. A single i. v. injection for a 30 g mouse was carried out at a standard dose of 1.88 mg/kg siRNA and 14.5 mg/kg lipid.

### EXAMPLE 4

### Delivery of Formulated siRNAs to the Tumor Endothelium

The purpose of this experiment was to analyze the applicability of formulated siRNA for cancer therapeutic intervention. For this purpose, the 19-mer siRNA described in example 2 was used and these molecules were formulated with cationic liposomes into siRNA-lipoplexes as described in example 3, and the in vivo applicability for tumor therapy was tested in the current study employing different tumor xenograft models (PC-3, HuH-7, 3Y1-RasV12).

The reaction conditions were as follows. Lipolexed siRNA-Cy3 was administered in tumor-bearing mice after i.v. administration by single i.v. injection. Tumor tissue sections of 4 hours post injection were analyzed by microscopy. The results are shown in FIG. 1: endothelial cells of different established tumors were targeted with siRNA-Cy3-lipoplexes (arrows). Uptake was studied in five sections in at least two independent xenograft experiments for each tumor type. Upper row shows fluorescent images of sections from subcutaneously grown PC-3 tumor (left panel) and Ras^{V12} transformed 3Y1 rat fibroblast tumor (middle panel) or intrahepatically grown HuH-7 tumor (right panel). Lower row, detection of delivered siRNA-Cy3 in endothelial cells of HuH-7 tumor. The tumor endothelial cells are shown by H+E (hematoxylin/eosin staining; left panel) characterized by their thin cytoplasm and the prominent nucleus (arrow). Consecutive sections show corresponding siRNA-Cy3 fluorescence (red, middle panel) and anti-CD34 immunostaining of the endothelial cells (right panel), respectively.

In three experimental tumor xenografts (two subcutaneously, s.c., and one intrahepatic, i.hep.) we detected significant fluorescence signals in the tumor vasculature (FIG. 1, upper panels). In contrast, equivalent amounts of non-formulated siRNAs administered by i.v. injection were not delivered to tumor vessels at all (data not shown). Lipoplexed siRNA-Cy3 uptake by the endothelial layer of the tumor vasculature (HuH-7, i.hep.) was confirmed by counterstaining with anti-CD34 antibody, an endothelial cell marker (FIG. 1, lower panels). Uptake of the intact siRNA-lipoplex by the endothelium was additionally confirmed using fluorescently labeled lipids ((Santel et al., 2006), data not shown). Taken together, these data demonstrate that cationic lipid based formulations of siRNAs allow for a predominant uptake of siRNAs into endothelial cells of blood vessels in liver and tumor.

### EXAMPLE 5

### In Vivo Gene Silencing of CD31 and its Effect on Tumor Growth

CD31 (platelet-endothelial-cell adhesion molecule 1 (PECAM-1)) was chosen as a suitable target to demonstrate in vivo siRNA mediated gene silencing directly since its expression is restricted primarily to endothelial cells. In addition the effect of CD31 loss of function on tumor growth was investigated. Screening of the 2′-O-methyl modified siRNA molecules described in example 2 (Table 1) in mouse and human derived endothelial cell lines (HUVEC, EOMA) led to the identification of several potent human and mouse specific CD31-siRNA molecules (FIG. 2a). The siRNA molecules chosen for the therapeutic approach comprised a specific siRNA^{CD31-8} and siRNA^{PTEN} as well as an unrelated siRNA sequence (Luciferase specific siRNA^{Luc}) as control molecules (we refer to siRNA^{CD31-8} when mentioning siRNA^{CD31} in the text below) (FIG. 2b).

The results are shown in FIG. 2. indicating the inhibition of CD31 expression in the tumor vasculature. (a) Identification of potent stabilized siRNAs for efficacious CD31 knockdown. HUVEC and murine EOMA cells were transfected with four different human, mouse specific siRNAs targeting CD31 (CD31-1, -2, -6, -8) and a control PTEN-siRNA. Specific protein knockdown was assessed by immunoblotting using anti-CD31 and anti-PTEN demonstrating highest efficacy of the siRNA^{CD31-8} molecule. (b) In vitro quality control and efficacy testing of lipoplexed siRNA used for systemic treatment in HUVEC. Immunoblotting using anti-CD31 antibody revealed a concentration dependent knockdown of CD31 in the case of siRNA^{CD31-8}, but not with control siRNA^{PTEN}. Reduction of CD31 had no effect on PI 3-kinase signaling as revealed by monitoring Akt phosphorylation status (P*-Akt), in contrast to the siRNA^{PTEN} control. CD34 protein level was not affected. (c) Effects of siRNA^{PTEN}- and siRNA^{CD31}-lipoplex treatment on liver enzymes (AST, ALT) measured at 24 h or 72 h post final i.v. treatment. Immune competent mice were treated for 6 consecutive days with daily doses of 1.88 mg/kg siRNA, 14.5 mg/kg lipid. Mean values (±s.e.m.) from mice (n=7) are shown. (d) Systemic siRNA-lipoplex treatment does not increase interferon-α serum-levels in immune competent mice. Male C57BL/6 mice received a single injection of poly(I:C) or indicated siRNA-lipoplex solutions (see dose above). Blood was collected 24 h post injection and IFNα levels were measured by ELISA.

To summarize, the siRNA^{CD31}- and siRNA^{PTEN}-lipoplexes used for the in vivo efficacy studies were tested in a dose dependent transfection experiment in HUVEC prior to the in vivo experiment. Representative immunoblots demonstrating the functionality and potency of these siRNA-lipoplexes are shown in FIG. 2b. Knockdown of CD31 protein was achieved with siRNA^{CD31} in the low nanomolar range with these formulations. Specificity of the siRNA^{CD31} mediated gene silencing was demonstrated by probing for PTEN, phosphorylated Akt and CD34. Unlike transfections with siRNA^{PTEN}, the phosphorylation status of Akt was not affected in HUVEC cells by reduction in CD31. CD34 protein level was not changed with both lipoplexes when compared to untreated controls. Treatment with the siRNA^{Luc}-lipoplexes had no effect on the expression of the two target genes CD31 and PTEN (data not shown).

Next, we established a dosing regimen, which allowed for repeated systemic treatment of mice using different lipoplex daily doses. Different total doses were achieved by administration of daily or bi-daily tail vein injections of 200 μl lipoplex solution (single dose 1.88 mg/kg siRNA; 14.5 mg/kg lipid). We did not observe severe toxic effects on the animal health status after repeated dosing as assessed by monitoring changes in levels of liver enzymes AST (aspartate aminotransferase) and ALT (alanine aminotransferase) (FIG. 2c) or body weight as an overall marker of general health (FIG. 4a, lower panels). The AST and ALT enzymes appear to be slightly increasing in the siRNA^{PTEN} treatment group, but this effect seems to be reversible over time. Moreover, in contrast to poly (I:C)-lipoplex (poly-inosinic-polycytidilic acid) no induction of the cytokine interferon-alpha (IFN-alpha) in blood from animals treated with three different siRNA-lipoplexes was detected (FIG. 3d) suggesting the absence of an unspecific immune response due to the application of siRNA-lipoplexes. Taken together these data suggest that siRNA-lipoplexes can be administered repeatedly without severe unspecific toxic side effects.

Subsequently, we analyzed the two dosing regimens representing either daily or bi-daily i.v. treatments in an efficacy study of siRNA^{CD31}-lipoplex on tumor growth inhibition.

The experimental conditions were as follows. FIG. 3 a shows the inhibition of s.c. xenograft tumor growth by siRNA^{CD31}-lipoplex treatment. Growth of established PC-3 xenografts was significantly inhibited with siRNA^{CD31}-lipoplex (diamonds) in comparison to siRNA^{Luc}-lipoplex (triangles) treated as indicated (standard dose 1.88 mg/kg/d siRNA; 14.5 mg/kg/d lipid; arrow) or isotonic sucrose (solid spheres). Changes in body weights were monitored during the treatment as shown in corresponding diagram below. A 3Y1-Ras^{V12} tumor xenograft was established in nude mice (7 mice per group). Growth of established 3Y1-Ras^{V12} tumors was significantly inhibited by siRNA^{CD31}-lipoplex (diamonds) when compared to siRNA^{PTEN}-lipoplexes (triangles) or isotonic sucrose (solid spheres). Bidaily treatment regimen (single standard dose) is indicated by double arrows. Data represent the means of daily tumor volume±s.e.m.; statistical significance is indicated by asterisk. Changes in body weights upon treatment are shown in the bottom panel. FIG. 3b shows the CD31 protein knockdown in 3Y1-Ras^{V12} tumor bearing mice treated systemically with siRNA^{CD31}-lipoplexes was confirmed by immunoblot analysis with extracts from tumor using anti-CD31 antibody and anti-PTEN as well as anti-CD34. As depicted in FIG. 3c CD31, protein reduction was directly assessed by immunostaining with anti-CD31 antibody in tumor sections from mice treated with isotonic sucrose, siRNA^{CD31}-lipoplex, and siRNA^{PTEN}-lipoplex. Consecutive sections were stained with anti-CD31 and anti-CD34 antibodies, respectively, to visualize the tumor vasculature. Reduced staining intensity for CD31, but not for CD34, was found in tumor sections from mice treated with siRNA^{CD31}-lipoplex. MVD quantification was determined by counting number (lower diagram) and total lengths (upper diagram) of CD31 or CD34 positive vessels, respectively.

To summarize, both treatment regimens resulted in a clear inhibitory effect on tumor growth. Systemic treatment of a slow growing s.c. PC-3 tumor xenograft with lipoplexed siRNA^{CD31} caused a significant delay in tumor growth in contrast to the siRNA^{Luc} and siRNA^{PTEN} controls (FIG. 3a, left diagram). No toxic side effects were observed during the treatment as assessed by body weight measurement. The growth of an established, fast growing 3Y1-Ras^{V12} s.c. xenograft was also inhibited by bi-daily i.v. treatments with lipoplexed siRNA^{CD31} without any toxic effects (FIG. 3a, right diagram). The observed inhibition was statistically significant when compared to the siRNA^{PTEN}-lipoplex as well as the sucrose treated control groups. Remarkably, a significant reduction of CD31 protein levels was detected in the 3Y1-tumor lysates from mice treated with siRNA^{CD31}-lipoplexes for two consecutive days in contrast to the unchanged protein levels observed in the control mice (FIG. 3b). To test for specificity and equal loading we analyzed in parallel the protein levels of CD34, another endothelial cell marker protein, as well as PTEN in these lysates. Furthermore, the reduction in CD31 expression was also revealed in situ, by measuring differences in the microvessel density (MVD) for the endothelial markers CD31 and CD34 in a xenograft tumor mouse model. MVD measurement is a surrogate marker for tumor angiogenesis, and analyzed by immunohistochemical staining of blood vessels with CD31 or CD34 specific antibodies (Fox and Harris, 2004; Uzzan et al., 2004; Weidner et al., 1991). MVD was compared between consecutive sections after immunostaining with CD31 and CD34 antibodies, respectively. The mice treated with the lipoplexed siRNA^{CD31} showed a statistically significant decrease in the total amount of CD31 positive vessels as measured by total number of vessels as well as vessel length (FIG. 3c). Staining with CD34 specific antibodies did not reveal a change in MVD indicating again specific CD31 silencing. Both control groups, siRNA^{PTEN} and isotonic sucrose treated, did not show differences in MVD assessment by either CD31 or CD34 staining. This result along with the molecular data on protein knockdown indicates the specific reduction in CD31 expression upon siRNA^{CD31}-lipoplex treatment.

### EXAMPLE 6

### Efficacy of Systemically Administered siRNA^{CD31}-Lipoplex in an Orthotopic Tumor Model

The potential therapeutic effect of the systemically administered siRNA^{CD31}-lipoplex was also investigated in mice bearing an orthotopic PC-3 tumor xenograft (Czaudema et al., 2003b; Stephenson et al., 1992). This seems to be a more clinical relevant model for human prostate cancer to corroborate the therapeutic potential of the siRNA^{CD31}-lipoplex treatment. For this orthotopic tumor model human PC-3 prostate cancer cells were directly implanted into the mouse prostate and the mice were sacrificed and analyzed for tumor and lymph node metastasis volumes 50 days after implantation.

The experimental conditions were as follows. The experimental design and treatment schedule is shown in FIG. 4a, more specifically the experimental design to analyze the efficacy of siRNA^{CD31}-lipoplex treatment in an orthotopic PC-3 prostate tumor and lymph node metastasis model. FIG. 4b shows the inhibition of volume from prostate PC-3 tumor and lymph node metastases in mice after treatment with the indicated siRNA-lipoplexes or sucrose. The tumor and metastasis volumes before treatment start are indicated on the left (d35, control). Statistical significance is indicated by asterisk. FIG. 5c shows the reduction of CD31 and Tie2 mRNA levels in mice treated with corresponding siRNA-lipoplexes in contrast to the control groups (sucrose, siRNA^{Luc}-lipoplex) as revealed by quantitative TaqMan RT-PCR after. The relative averaged amount of mRNAs obtained from nine mice is shown for CD31, Tie2 and the CD34 control.

To summarize, the negative control siRNA^{Luc}-lipoplex but also the siRNA^{Tie2}-lipoplex showed only some minor but no statistically significant reduction in tumor and metastasis volume when compared to the sucrose control group (FIG. 4b). However, a highly significant siRNA^{CD31} specific tumor growth inhibition as well as a reduction in the volume of lymph node metastases is observed upon systemic treatment with the lipoplexed siRNA^{CD31} (FIG. 4b). A comparison with the pretreatment control group (9 randomized mice sacrificed on day 35) indicates that additional growth of both tumor and metastasis is observed upon siRNA^{Luc}- and siRNA^{Tie2}-lipoplex treatment but not in the mice treated with the siRNA^{CD31}. We intended to determine target mRNA expression levels by quantitative RT-PCR in endothelial cells of the tumor tissue, but were not able to precisely quantify mRNA levels probably due to the very low amount of mRNA derived from vasculature of this specific tumor type. Therefore, we focused on measuring changes in the expression level for CD31, Tie2 and CD34 in lung tissues from corresponding mice of the different treatment groups used in the efficacy experiment (FIG. 4b). We have previously observed that lung endothelium can be efficiently targeted by this technology and that CD31 mRNA knockdown can be experimentally demonstrated in this tissue (see (Santel et al., 2006)). Mice treated with siRNA^{Tie2}- and siRNA^{C31}-lipoplexes showed significant reduction of corresponding mRNA levels in a sequence-specific manner demonstrating the functionality of the applied siRNA-lipoplexes in the PC-3 orthotopic efficacy study (FIG. 4c). In contrast, control mice treated with siRNA^{Luc}-lipoplex showed no inhibition in CD31 and Tie2 levels. In addition, the amount of the endothelia-specifically expressed gene CD34 was not affected in any treatment group. Taken together, both in vivo xenograft experiments demonstrate that tumor/metastasis growth is selectively suppressed by repeated systemic administration of siRNA^{CD31}-lipoplexes. These data imply that CD31 (PECAM-1), a non-classical drug target, might be a suitable gene target for RNAi-based anti-angiogenic therapeutic intervention.

### EXAMPLE 7

### Comparing Target Specificity of a 23mer siRNA with the One of a 19mer siRNA

The purpose of this experiment was to provide evidence that human 23mer siRNA^{CD31-8} showed the same efficacy as the corresponding 19mer siRNA^{CD31}-8.

The experimental procedure basically corresponds to the one outlined in the above examples. More specifically, HUVECs were transfected with the respective siRNAs at 20 nM with AtuFECT01. Protein knockdown was assessed by Western blot 72 hours post transfection. The result thereof is indicated in FIG. 6.

As may be taken from the Western blot, the siRNA specifically directed against human CD31 and having either a length of 23 base pairs or 19 base pairs is highly effective in knocking down CD31. The molecules and single strands thereof used are also specified in FIG. 6, whereby the siRNA molecule specifically directed against the human sequence of CD31 comprising 23 base pairs is referred to as CD31_{—}8_h_{—}23mer, and the siRNA molecule specifically directed against both the human and the mouse sequence of CD31 comprising 19 base pairs is referred to as CD31_{—}8_hm_{—}19mer.

In any case, the character “h” indicates that the sequence is specific for the human sequence of the target mRNA, whereas the characters “hm” indicate that the sequence is specific for both the mouse and the human sequence of the target mRNA. The nucleotides printed in bold are 2-O′-Me-modified.

In the Western blot shown in FIG. 5 siRNA^{Luc} served as a negative control (ut: untreated). The particular sequences of this luciferase specific siRNA molecules are as follows:

Luc-siRNA-1B
Cguacgcggaauacuucga
Luc-siRNA-1A
ucgaaguauuccgcguacg

Apart from providing experimental evidence that both a 19mer and a 23 mer double-stranded nucleic acid as specified herein is effective in knocking down CD31 mRNA, species specificity is also shown. For such purpose, the human 23-mer is compared with corresponding 23-mer for the mouse and rat homolog, respectively. The respective strands are also indicated in FIG. 5, whereby CD31-8-m-23-A indicates the antisense strand (in 5′->3′-direction), CD31-8-m-23-B indicates the mouse sense strand (indicated in 5′->3′-direction), CD31-8-r-23-A indicates the rat antisense strand (indicated in 5′->3′-direction and CD31-8-r-23-B indicates the rat sense strand (indicated in 5′->3′-direction)

### REFERENCES

To the extent it is referred herein to various documents of the prior art, such documents the complete bibliographic data of which read as follows, are incorporated herein in their entirety by reference.
Czauderna, F., Fechtner, M., Dames, S., Aygun, H., Klippel, A., Pronk, G. J., Giese, K. and Kaufmann, J. (2003a) Structural variations and stabilising modifications of synthetic siRNAs in mammalian cells. Nucleic Acids Res, 31, 2705-2716.
Czauderna, F., Santel, A., Hinz, M., Fechtner, M., Durieux, B., Fisch, G., Leenders, F., Arnold, W., Giese, K., Klippel, A. and Kaufmann, J. (2003b) Inducible shRNA expression for application in a prostate cancer mouse model. Nucleic Acids Res, 31, e127.
Fox, S. B. and Harris, A. L. (2004) Histological quantitation of tumour angiogenesis. Apmis, 112, 413-430.
Klippel, A., Escobedo, J. A., Hirano, M. and Williams, L. T. (1994) The interaction of small domains between the subunits of phosphatidylinositol 3-kinase determines enzyme activity. Mol Cell Biol, 14, 2675-2685.
Leenders, F., Mopert, K., Schmiedeknecht, A., Santel, A., Czauderna, F., Aleku, M., Penschuck, S., Dames, S., Sternberger, M., Rohl, T., Wellmann, A., Arnold, W., Giese, K., Kaufmann, J. and Klippel, A. (2004) PKN3 is required for malignant prostate cell growth downstream of activated PI 3-kinase. Embo J, 23, 3303-3313.
Santel, A., Aleku, M., Keil, O., Endruschat, J., Esche, V., Fisch, G., Dames, S., Loffler, K., Fechtner, M., Arnold, W., Giese, K., Klippel, A. and Kaufmann, J. (2006) A novel siRNA-lipoplex technology for RNAi in the mouse vascular endothelium. Gene Ther, 13, 1222-1234
Stambolic, V., Suzuki, A., de la Pompa, J. L., Brothers, G. M., Mirtsos, C., Sasaki, T., Ruland, J., Penninger, J. M., Siderovski, D. P. and Mak, T. W. (1998) Negative regulation of PKB/Akt-dependent cell survival by the tumor suppressor PTEN. Cell, 95, 29-39.
Stephenson, R. A., Dinney, C. P., Gohji, K., Ordonez, N. G., Killion, J. J. and Fidler, I. J. (1992) Metastatic model for human prostate cancer using orthotopic implantation in nude mice. J Natl Cancer Inst, 84, 951-957.
Sternberger, M., Schmiedeknecht, A., Kretschmer, A., Gebhardt, F., Leenders, F., Czauderna, F., Von Carlowitz, I., Engle, M., Giese, K., Beigelman, L. and Klippel, A. (2002) GeneBlocs are powerful tools to study and delineate signal transduction processes that regulate cell growth and transformation. Antisense Nucleic Acid Drug Dev, 12, 131-143.
Uzzan, B., Nicolas, P., Cucherat, M. and Perret, G. Y. (2004) Microvessel density as a prognostic factor in women with breast cancer: a systematic review of the literature and meta-analysis. Cancer Res, 64, 2941-2955.
Vermeulen, P. B., Verhoeven, D., Hubens, G., Van Marck, E., Goovaerts, G., Huyghe, M., De Bruijn, E. A., Van Oosterom, A. T. and Dirix, L. Y. (1995) Microvessel density, endothelial cell proliferation and tumour cell proliferation in human colorectal adenocarcinomas. Ann Oncol, 6, 59-64.
Weidner, N., Semple, J. P., Welch, W. R. and Folkman, J. (1991) Tumor angiogenesis and metastasis—correlation in invasive breast carcinoma. N Engl J Med, 324, 1-8.

The features of the present invention disclosed in the specification, the claims, the sequence listing and/or the drawings may both separately and in any combination thereof be material for realizing the invention in various forms thereof.

1-33. (canceled)

34. A nucleic acid molecule comprising a double-stranded structure,
wherein the double-stranded structure comprises a first strand and a second strand,
wherein the first strand comprises a first stretch of contiguous nucleotides and said first stretch is at least partially complementary to a target nucleic acid, and
wherein the second strand comprises a second stretch of contiguous nucleotides and said second stretch is at least partially complementary to the first stretch,
wherein the first stretch comprises a nucleic acid sequence which is at least complementary to a nucleotide core sequence of the nucleic acid sequence according to SEQ ID NO: 1,
wherein the nucleotide core sequence comprises the nucleotide sequence
from nucleotide positions 1277 to 1295 of SEQ ID NO: 1;
from nucleotide positions 2140 to 2158 of SEQ ID NO: 1;
from nucleotide positions 2391 to 2409 of SEQ ID NO: 1; and
wherein the first stretch is additionally at least partially complementary to a region preceding the 5′ end of the nucleotide core sequence and/or to a region following the 3′ end of the nucleotide core sequence.

35. The nucleic acid according to claim 34, wherein the first stretch is complementary to the nucleotide core sequence.

36. The nucleic acid according to claim 34, wherein the first stretch is additionally complementary to the region following the 3′ end of the nucleotide core sequence.

37. The nucleic acid according to claim 34, wherein the first stretch is complementary to the target nucleic acid over 18 to 29 nucleotides.

38. The nucleic acid according to claim 37, wherein the nucleotides are consecutive nucleotides.

39. The nucleic acid according to claim 34, wherein the first stretch and/or the second stretch comprises from 18 to 29 consecutive nucleotides.

40. The nucleic acid according to claim 34, wherein the first strand consists of the first stretch and/or the second strand consists of the second stretch.

41. The nucleic acid according to claim 34 comprising a double-stranded structure, wherein the double-stranded structure is formed by a first strand and a second one strand, wherein the first strand comprises a first stretch of contiguous nucleotides and the second strand comprises a second stretch of contiguous nucleotides and wherein said first stretch is at least partially complementary to said second stretch, wherein
the first stretch consists of a nucleotide sequence according to SEQ ID NO: 2 and the second stretch consists of a nucleotide sequence according to SEQ ID NO: 3;
the first stretch consists of a nucleotide sequence according to SEQ ID NO: 4 and the second stretch consists of a nucleotide sequence according to SEQ ID NO: 5;
the first stretch consists of a nucleotide sequence according to SEQ ID NO: 6 and the second stretch consists of a nucleotide sequence according to SEQ ID NO: 7;
the first stretch consists of a nucleotide sequence according to SEQ ID NO: 8 and the second stretch consists of a nucleotide sequence according to SEQ ID NO: 9.

42. The nucleic acid according to claim 34, wherein the first stretch and/or the second stretch comprises a plurality of groups of modified nucleotides having a modification at the 2′ position, wherein within the stretch each group of modified nucleotides is flanked on one or both sides by a flanking group of nucleotides, wherein the flanking nucleotide(s) forming the flanking group of nucleotides is/are either an unmodified nucleotide or a nucleotide having a modification different from the modification of the modified nucleotides, wherein the first stretch and/or the second stretch comprises at least two groups of modified nucleotides and at least two flanking groups of nucleotides.

43. The nucleic acid according to claim 34, wherein the first stretch and/or the second stretch comprises a pattern of groups of modified nucleotides and/or a pattern of flanking groups of nucleotides, wherein the pattern is a positional pattern.

44. The nucleic acid according to claim 34, wherein the first stretch and/or the second stretch comprise at the 3′ end a dinucleotide, wherein such dinucleotide is TT.

45. The nucleic acid according to claim 44, wherein the length of the first stretch and/or of the second stretch consists of 19 to 23 nucleotides.

46. The nucleic acid according to claim 34, wherein the first and/or the second stretch comprise an overhang of 1 to 5 nucleotides at the 3′ end.

47. The nucleic acid according to claim 46, wherein the length of the double-stranded structure is from about 16 to 24 nucleotide pairs.

48. The nucleic acid according to claim 34, wherein the first strand and the second strand are covalently linked to each other.

49. A lipoplex comprising a nucleic acid according to claim 34 and a liposome.

50. The lipoplex according to claim 49, wherein the liposome consists of
a) about 50 mol % β-arginyl-2,3-diaminopropionic acid-N-palmityl-N-oleyl-amide trihydrochloride, or (β-(L-arginyl)-2,3-L-diaminopropionic acid-N-palmityl-N-oleyl-amide tri-hydrochloride);
b) about 48 to 49 mol % 1,2-diphytanoyl-sn-glycero-3-phosphoethanolamine (DPhyPE); and
c) about 1 to 2 mol % 1,2-distearoyl-sn-glycero-3-phosphoethanolamine-polyethylen-glycole or N-(Carbonyl-methoxypolyethyleneglycol-2000)-1,2-distearoyl-sn-glycero-3-phosphoethanolamine sodium salt.

51. The lipoplex according to claim 50, wherein the zeta-potential of the lipoplex is about 40 to 55 mV.

52. The lipoplex according to claim 50, wherein the lipoplex has a size of about 80 to 200 nm, of about 100 to 140 nm or of about 110 nm to 130 nm, as determined by QELS.

53. A vector comprising or coding for a nucleic acid according to claim 34.

54. An isolated cell comprising a nucleic acid according to claim 34.

55. A pharmaceutical composition comprising:
a) a nucleic acid according to claim 34;
b) a lipoplex comprising a nucleic acid according to claim 34;
c) a vector comprising a nucleic acid according to claim 34; or
d) a cell comprising a nucleic acid according to claim 34.

56. The composition according to claim 55, wherein the composition is a pharmaceutical composition comprising a pharmaceutically acceptable vehicle.

57. A method of treating an angiogenesis-dependent disease, or diseases characterized or caused by insufficient, abnormal or excessive angiogenesis comprising the administration of a pharmaceutical composition according to claim 55 to a subject in need of treatment.

58. The method according to claim 57, wherein the angiogenesis is angiogenesis of adipose tissue, skin, heart, eye, lung, intestines, reproductive organs, bone and joints.

59. The method according to claim 57, wherein the disease is selected from infectious diseases, autoimmune disorders, vascular malformation, atherosclerosis, transplant arteriopathy, obesity, psoriasis, warts, allergic dermatitis, persistent hyperplastic vitrous syndrome, diabetic retinopathy, retinopathy of prematurity, age-related macular disease, choroidal neovascularization, primary pulmonary hypertension, asthma, nasal polyps, inflammatory bowel and periodontal disease, ascites, peritoneal adhesions, endometriosis, uterine bleeding, ovarian cysts, ovarian, ovarian hyperstimulation, arthritis, synovitis, osteomyelitis or osteophyte formation.

60. A method for the treatment of a neoplastic disease, cancer or a solid tumor comprising the administration of a composition according to claim 55 to a subject in need of treatment.

61. The method according to claim 60, wherein the disease is bone cancer, breast cancer, prostate cancer, cancer of the digestive system, colorectal cancer, liver cancer, lung cancer, kidney cancer, urogenital cancer, pancreatic cancer, pituitary cancer, testicular cancer, orbital cancer, head and neck cancer, cancer of the central nervous system or cancer of the respiratory system.

62. The method according to claim 60, further comprising the administration of one or more additional therapies selected from chemotherapy, cryotherapy, hyperthermia, antibody therapy or radiation therapy.


SEQUENCE LISTING (11 sequences)

SEQ ID NO: 1 - RNA, 3223 nt, Homo sapiens
  mRNA (1)..(3223): mRNA of CD31
ccaggccccauuguucccgguuuccagccauggcugccauuaccugaccagcgccacagccggucucucugcaggcgccgggagaagugaccagagcaauuucugcuuuucacagggcggguuucucaacggugacuugugggcagugccuucugcugagcgagucauggcccgaaggcagaacuaacugugccugcagucuucacucucaggaugcagccgaggugggcccaaggggccacgauguggcuuggaguccugcugacccuucugcucuguucaagccuugagggucaagaaaacucuuucacaaucaacaguguugacaugaagagccugccggacuggacggugcaaaaugggaagaaccugacccugcagugcuucgcggaugucagcaccaccucucacgucaagccucagcaccagaugcuguucuauaaggaugacgugcuguuuuacaacaucuccuccaugaagagcacagagaguuauuuuauuccugaaguccggaucuaugacucagggacauauaaauguacugugauugugaacaacaaagagaaaaccacugcagaguaccagguguugguggaaggagugcccagucccagggugacacuggacaagaaagaggccauccaaggugggaucgugagggucaacuguucugucccagaggaaaaggccccaauacacuucacaauugaaaaacuugaacuaaaugaaaaaauggucaagcugaaaagagagaagaauucucgagaccagaauuuugugauacuggaauuccccguugaggaacaggaccgcguuuuauccuuccgaugucaagcuaggaucauuucugggauccauaugcagaccucagaaucuaccaagagugaacuggucaccgugacggaauccuucucuacacccaaguuccacaucagccccaccggaaugaucauggaaggagcucagcuccacauuaagugcaccauucaagugacucaccuggcccaggaguuuccagaaaucauaauucagaaggacaaggcgauuguggcccacaacagacauggcaacaaggcuguguacucagucauggccaugguggagcacaguggcaacuacacgugcaaaguggaguccagccgcauauccaaggucagcagcaucguggucaacauaacagaacuauuuuccaagcccgaacuggaaucuuccuucacacaucuggaccaaggugaaagacugaaccuguccugcuccaucccaggagcaccuccagccaacuucaccauccagaaggaagauacgauugugucacagacucaagauuucaccaagauagccucaaagucggacagugggacguauaucugcacugcagguauugacaaaguggucaagaaaagcaacacaguccagauagucguaugugaaaugcucucccagcccaggauuucuuaugaugcccaguuugaggucauaaaaggacagaccaucgaaguccguugcgaaucgaucaguggaacuuugccuauuucuuaccaacuuuuaaaaacaaguaaaguuuuggagaauaguaccaagaacucaaaugauccugcgguauucaaagacaaccccacugaagacgucgaauaccaguguguugcagauaauugccauucccacgccaaaauguuaagugagguucugagggugaaggugauagccccgguggaugagguccagauuucuauccugucaaguaaggugguggagucuggagaggacauugugcugcaaugugcugugaaugaaggaucuggucccaucaccuauaaguuuuacagagaaaaagagggcaaacccuucuaucaaaugaccucaaaugccacccaggcauuuuggaccaagcagaaggcuaacaaggaacaggagggagaguauuacugcacagccuucaacagagccaaccacgccuccaguguccccagaagcaaaauacugacagucagagucauucuugccccauggaagaaaggacuuauugcagugguuaucaucggagugaucauugcucucuugaucauugcggccaaauguuauuuucugaggaaagccaaggccaagcagaugccaguggaaauguccaggccagcaguaccacuucugaacuccaacaacgagaaaaugucagaucccaauauggaagcuaacagucauuacggucacaaugacgaugucggaaaccaugcaaugaaaccaauaaaugauaauaaagagccucugaacucagacgugcaguacacggaaguucaaguguccucagcugagucucacaaagaucuaggaaagaaggacacagagacaguguacagugaaguccggaaagcugucccugaugccguggaaagcagauacucuagaacggaaggcucccuugauggaacuuagacagcaaggccagaugcacaucccuggaaggacauccauguuccgagaagaacagauaaucccuguauuucaagaccucugugcacuuauuuaugaaccugcccugcucccacagaacacagcaauuccucaggcuaagcugccgguucuuaaauccauccugcuaaguuaauguuggguagaaagagauacagaggggcuguugaauuucccacauacccuccuuccaccaaguuggaacauccuuggaaauuggaagagcacaagaggagauccagggcaaggccauugggauauucugaaacuugaauauuuuguuuugugcagagauaaagaccuuuuccaugcacccucauacacagaaaccaauuuucuuuuuuauacucaaucauuucuagcgcauggccugguuagaggcugguuuuuucucuuuuccuuugguccuucaaaggcuuguaguuuuggcuaguccuuguucuuuggaaauacacagugcugaccagacagccucccccuguccccucuaugaccucgcccuccacaaaugggaaaaccagacuacuugggagcaccuccugugaaauaccaaccugaagacaccguucauucaggcaacgcacaaaacagaaaaugaagguggaacaagcacagauguucuucaacuguuuuugucuacacucuuucucuuuuccucuaccaugcugaaggcugaaagacaggaagauggugccaucagcaaauauuauucuuaauugaaaacuugaaaaaaaaaaaaaaaaaaaaaaaaaaaaaa

SEQ ID NO: 2 - RNA, 19 nt, Artificial
  siRNA
uuccguucuagaguaucug

SEQ ID NO: 3 - RNA, 19 nt, Artificial
  siRNA
cagauacucuagaacggaa

SEQ ID NO: 4 - RNA, 19 nt, Artificial
  siRNA
uguuggaguucagaagugg

SEQ ID NO: 5 - RNA, 19 nt, Artificial
  siRNA
ccacuucugaacuccaaca

SEQ ID NO: 6 - RNA, 19 nt, Artificial
  siRNA
uucuggauggugaaguugg

SEQ ID NO: 7 - RNA, 19 nt, Artificial
  siRNA
ccaacuucaccauccagaa

SEQ ID NO: 8 - RNA, 23 nt, Artificial
  siRNA
uuccguucuagaguaucugcuuu

SEQ ID NO: 9 - RNA, 23 nt, Artificial
  siRNA
aaagcagauacucuagaacggaa

SEQ ID NO: 10 - RNA, 19 nt, Artificial
  siRNA
ucgaaguauuccgcguacg

SEQ ID NO: 11 - RNA, 19 nt, Artificial
  siRNA
cguacgcggaauacuucga